(19) United States
(12) Patent Application Publication
(10) Pub. No.: US 2026/0117329 A1
(43) Pub. Date: Apr. 30, 2026

(54) UTILIZATION OF dITP FOR PREFERENTIAL/SELECTIVE AMPLIFICATION OF RNA VERSUS DNA TARGETS BASED ON STRAND-SEPARATION TEMPERATURE

(71) Applicant: Roche Molecular Systems, Inc., Pleasanton, CA (US)

(72) Inventors: Kristina Chu, Pleasanton, CA (US); Barbara Eckert, El Cerrito, CA (US); Aaron Thaddeus Hamilton, Mountain House, CA (US); Thomas W. Myers, Mountain View, CA (US); Jingtao Sun, San Ramon, CA (US); Ling Wang, Dublin, CA (US)

(21) Appl. No.: 17/594,793

(22) PCT Filed: May 1, 2020
(86) PCT No.: PCT/EP2020/062182

§ 371 Date: Mar. 30, 2022

Related U.S. Application Data
(60) Provisional application No. 62/842,222, filed on May 2, 2019.

(51) Int. Cl. C12Q 1/70

(52) U.S. Cl. CPC C12Q 1/706

(57) ABSTRACT

Methods for preferentially and/or selectively detecting and/or quantitating target RNA, over DNA, in a biological or non-biological sample are described. The methods include performing an amplifying step, a hybridizing step, and a detecting step. Furthermore, the reaction mixture includes dNTPs, that include dITP. Also described are kits, reaction mixtures, and oligonucleotides (e.g., primer and probe) for the preferential and/or selective amplification and detection and/or quantitation of target RNA (over DNA), in the presence of dITP. The methods, kits, and reaction mixtures are for the preferential and/or selective detection and/or quantitation of RNA, over DNA, and are particularly useful with samples that comprise a number of different types of nucleic acids (e.g., DNA and RNA).

### FIELD OF THE INVENTION

The present disclosure relates to the field of nucleic acid detection. Within this field, the present invention concerns the amplification, detection, and/or quantitation of a target nucleic acid that may be present in a sample and particularly, the selective and preferential amplification, detection, and/or quantitation of a target ribonucleic acid (RNA) versus deoxyribonucleic acid (DNA), utilizing 2′-deoxyinosine 5′-triphosphate (dITP) as a deoxynucleoside triphosphate (dNTP), along with primers and probes. The invention further provides reaction mixtures and kits containing dITP and primers and probes for the selective and preferential amplification and detection RNA versus DNA (for example of Hepatitis B Virus (HBV) RNA).

### BACKGROUND OF THE INVENTION

Many biological samples will include a number of different types of nucleic acids, including both deoxyribonucleic acid (DNA) and/or ribonucleic acid (RNA). In certain circumstances, it may be desirable or advantageous to selectively and preferentially detect only one type of nucleic acid (e.g., RNA and not DNA, or DNA and not RNA). For example, it may be desirable or advantageous to detect genomic DNA, while not detecting potentially interfering RNA that might be in the same biological sample. Alternatively, it might be desirable or advantageous to detect RNA, while not detecting any potentially interfering genomic DNA that might be in the same biological sample.

There are methods well known in the art for selectively and preferentially depleting undesirable nucleic acids or enriching desirable nucleic acids, from a biological sample. An example of an existing solution to, for example, deplete undesirable DNA from a biological sample, thereby improving the selectivity of RNA, in an amplification reaction (e.g., polymerase chain reaction (PCR), would be the addition of deoxyribonuclease (DNase) enzyme to sample prepared material prior to PCR cycling. In this case, the DNAse enzyme selectively and preferentially degrades only DNA, while leaving RNA intact, in the sample. However, there are drawbacks to this methodology. For example, this method runs the risk of introducing DNase into the reaction. Moreover, this method would require an extra step to eliminate the DNase in the samples prior to PCR cycling, so as to prevent degradation of oligonucleotides (e.g., primers, probes, and amplicons/amplified product) in the PCR by residual DNase. If the DNase is not eliminated, and is introduced into the PCR reaction, the oligonucleotides, such as primers, probes, and amplicons/amplified product, would be degraded and the PCR will not be successful. Yet another example of an existing solution for preferential and selective RNA amplification versus DNA, is to design a PCR assay around the poly-A tail region in the RNA. However, because all mRNA contain a poly-A tail, this method is not specific enough to detect specific RNA targets of interest.

In the field of molecular diagnostics, the amplification and detection of nucleic acids is of considerable significance. Such methods can be employed to detect any number of micro-organisms, such as viruses and bacteria. The most prominent and widely-used amplification technique is the Polymerase Chain Reaction (PCR). Other amplification techniques include Ligase Chain Reaction, Polymerase Ligase Chain Reaction, Gap-LCR, Repair Chain Reaction, 3SR, NASBA, Strand Displacement Amplification (SDA), Transcription Mediated Amplification (TMA), and Qβ-amplification. Automated systems for PCR-based analysis often make use of a real-time detection of product amplification during the PCR process in the same reaction vessel. Key to such methods is the use of modified oligonucleotides carrying reporter groups or labels. The present invention represents an improvement over the existing art. The present invention relates to a method of preferentially and/or selectively amplifying and detecting RNA (versus DNA), involving the utilization of 2′-deoxyinosine 5′-triphosphate (dITP) as a deoxynucleoside triphosphate (dNTP), in an amplification reaction. The utilization of dITP results in an alteration of the stand separation temperature, which allows for improved RNA selectivity of RNA targets that cannot be easily discerned from DNA targets or targeted by exploiting obvious biological properties such as the poly-A tail or intron/exon junctions. In this case, dITP behaves as a deoxyguanosine triphosphate (dGTP) analogue, sharing a similar chemical structure to dGTP, but with dITP lacking the 2-amino group, compared to dGTP (see, FIG. 1). This difference results in a decrease in base stacking interactions and hydrogen bonding, which leads to a reduction in Tₘ or strand separation temperature. Substitution of dGTP with dITP reduces the Tₘ of the first and second strand complementary DNA (cDNA) synthesis and subsequent newly formed amplicon which allows thermal cycling at lower temperatures, such that the strand separation temperature is below that required for melting of the native double-stranded DNA. Therefore, integrating dITP preferentially amplifies RNA by preventing strand separation of the double-stranded DNA, thereby preventing the polymerase from accessing and amplifying a DNA target. Thus, there remains a need in the art for a quick, reliable, and specific method of preferentially detecting and/or amplifying RNA targets in a sample that may contain a mixture of nucleic acid types (e.g., DNA and RNA).

The present invention provides a method for selective and/or preferential detection and/or amplification of RNA targets, by utilizing dITP. This is particularly useful in circumstances where a biological sample contain many different types of nucleic acid types (e.g., RNA and DNA), and it is desirable to detect only RNA, and not potentially interfering DNA.

### SUMMARY OF THE INVENTION

Certain embodiments in the present disclosure relate to methods for the preferential detection and/or quantification of target RNA over DNA in a biological or non-biological sample. Such methods may be performed in vitro. Embodiments include methods for preferential detection and/or quantification of target RNA over DNA, comprising performing at least one cycling step, which may include an amplifying step and a hybridizing step. Furthermore, embodiments include primers, probes, polymerase(s), dNTPs (including dATP, dTTP, dCTP, dGTP, and dITP), and kits that are designed for the preferential detection and/or quantification of target RNA over DNA. One embodiment is directed to a method for selectively detecting and/or quantitating one or more target RNA over DNA in a sample, the method comprising: (a) providing a sample; (b) providing one or more polymerases; (c) providing dNTPs, wherein the dNTPs comprise at least dITP; (d) performing an amplification step, wherein the amplification step comprises contacting the sample with one or more set of oligonucleotide primers specific for the target RNA; (e) performing a hybridizing step, wherein the hybridizing step comprises contacting the amplification product, if the target RNA is present in the sample, with one or more set of oligonucleotide probes; and (f) performing a detecting step, wherein the detecting step comprises detecting the presence or absence of the target RNA, wherein the presence of the amplification product is indicative of the presence of the target RNA in the sample, and wherein the absence of the amplification product is indicative of the absence of the target RNA in the sample; and wherein during the amplification step, the one or more polymerases amplify the target RNA, if present in the sample, by incorporating the dITP into the amplification product, thereby selectively detecting and/or quantitating target RNA as compared to DNA in a sample. In one embodiment, the dNTPs further comprise dATP, dTTP, dCTP, and dGTP. In one embodiment, the ratio of dITP:dGTP is 3:1, 1:1, or 1:3. In one embodiment, the ratio of dITP:dGTP is 3:1. In one embodiment, the dNTPs comprises equal amounts of: (i) dATP; (ii) dTTP; (iii) dCTP; and (iv) dGTP+dITP. In one embodiment, the ratio of dITP:dGTP is 3:1, 1:1, or 1:3. In one embodiment, the ratio of dITP:dGTP is 3:1. In one embodiment, the sample contains both RNA and DNA. In one embodiment, the sample is a biological sample. In one embodiment, the biological sample is blood, plasma, or urine. In one embodiment, the one or more polymerase is a DNA polymerase. In one embodiment, the presence of the dITP results in a reduction in the melting temperature (Tₘ) of the amplification product. In one embodiment, the target RNA is Hepatitis B Virus (HBV) RNA. In one embodiment, the one or more set of oligonucleotide primers and the one or more set of oligonucleotide probes hybridize to a nucleic acid sequence comprising SEQ ID NO:4. In one embodiment, the one or more set of oligonucleotide primers comprise oligonucleotide primers having a nucleic acid sequence of SEQ ID NOs:1 and 2, and wherein the one or more set of oligonucleotide probes comprise an oligonucleotide probe having a nucleic acid sequence of SEQ ID NO:3. In one embodiment, the HBV RNA is HBV pre-genomic RNA (pgRNA). In one embodiment, the HBV pgRNA is a surrogate for HBV covalently closed circular DNA (cccDNA). In one embodiment, the amount of HBV pgRNA quantitated is a factor considered for a treatment decision regarding a patient from whom the sample originates.

One embodiment is directed to a kit for selectively detecting and/or quantitating one or more target RNA over DNA in a sample, the kit comprising: (a) one or more polymerases; (b) dNTPs, wherein the dNTPs comprise at least dITP; (c) one or more set of oligonucleotide primers specific for the target RNA; and (d) one or more set of oligonucleotide probes. In one embodiment, the dNTPs further comprise dATP, dTTP, dCTP, and dGTP. In one embodiment, the ratio of dITP:dGTP is 3:1, 1:1, or 1:3. In one embodiment, the ratio of dITP:dGTP is 3:1. In one embodiment, the dNTPs comprises equal amounts of: (i) dATP; (ii) dTTP; (iii) dCTP; and (iv) dGTP+dITP. In one embodiment, the ratio of dITP:dGTP is 3:1, 1:1, or 1:3. In one embodiment, the ratio of dITP:dGTP is 3:1. In one embodiment, the sample contains both RNA and DNA. In one embodiment, the sample is a biological sample. In one embodiment, the biological sample is blood, plasma, or urine. In one embodiment, the one or more polymerase is a DNA polymerase. In one embodiment, the presence of the dITP results in a reduction in the melting temperature (Tₘ) of the amplification product. In one embodiment, the target RNA is Hepatitis B Virus (HBV) RNA. In one embodiment, the one or more set of oligonucleotide primers and the one or more set of oligonucleotide probes hybridize to a nucleic acid sequence comprising SEQ ID NO:4. In one embodiment, the one or more set of oligonucleotide primers comprise oligonucleotide primers having a nucleic acid sequence of SEQ ID NOs:1 and 2, and wherein the one or more set of oligonucleotide probes comprise an oligonucleotide probe having a nucleic acid sequence of SEQ ID NO:3. In one embodiment, the HBV RNA is HBV pre-genomic RNA (pgRNA). In one embodiment, the HBV pgRNA is a surrogate for HBV covalently closed circular DNA (cccDNA). In one embodiment, the amount of HBV pgRNA quantitated is a factor considered for a treatment decision regarding a patient from whom the sample originates.

Another embodiment is directed to a method for selectively detecting and/or quantitating one or more target HBV RNA over DNA in a sample, the method comprising: (a) providing a sample; (b) providing one or more polymerases; (c) providing dNTPs, wherein the dNTPs comprise at least dITP; (d) performing an amplification step, wherein the amplification step comprises contacting the sample with one or more set of oligonucleotide primers specific for the target HBV RNA, wherein the one or more set of oligonucleotide primers comprise oligonucleotide primers having a nucleic acid sequence of SEQ ID NOs:1 and 2; (e) performing a hybridizing step, wherein the hybridizing step comprises contacting the amplification product, if the target HBV RNA is present in the sample, with one or more set of oligonucleotide probes, wherein the one or more set of oligonucleotide probes comprise a oligonucleotide probe having a nucleic acid sequence of SEQ ID NO:3; and (f) performing a detecting step, wherein the detecting step comprises detecting the presence or absence of the target HBV RNA, wherein the presence of the amplification product is indicative of the presence of the target HBV RNA in the sample, and wherein the absence of the amplification product is indicative of the absence of the target HBV RNA in the sample; and wherein during the amplification step, the one or more polymerases amplify the target HBV RNA, if present in the sample, by incorporating the dITP into the amplification product, thereby selectively detecting and/or quantitating target RNA as compared to DNA in a sample. In one embodiment, the dNTPs further comprise dATP, dTTP, dCTP, and dGTP. In one embodiment, the ratio of dITP:dGTP is 3:1, 1:1, or 1:3. In one embodiment, the ratio of dITP:dGTP is 3:1. In one embodiment, the dNTPs comprises equal amounts of: (i) dATP; (ii) dTTP; (iii) dCTP; and (iv) dGTP+dITP. In one embodiment, the ratio of dITP:dGTP is 3:1, 1:1, or 1:3. In one embodiment, the ratio of dITP:dGTP is 3:1. In one embodiment, the sample contains both RNA and DNA. In one embodiment, the sample is a biological sample. In one embodiment, the biological sample is blood, plasma, or urine. In one embodiment, the one or more polymerase is a DNA polymerase. In one embodiment, the presence of the dITP results in a reduction in the melting temperature (Tₘ) of the amplification product. In one embodiment, the one or more set of oligonucleotide primers and the one or more set of oligonucleotide probes hybridize to a nucleic acid sequence comprising SEQ ID NO:4. In one embodiment, the HBV RNA is HBV pre-genomic RNA (pgRNA). In one embodiment, the HBV pgRNA is a surrogate for HBV covalently closed circular DNA (cccDNA). In one embodiment, the amount of HBV pgRNA quantitated is a factor considered for a treatment decision regarding a patient from whom the sample originates.

In one aspect, amplification can employ a polymerase enzyme having 5′ to 3′ nuclease activity. Thus, the donor fluorescent moiety and the acceptor moiety, e.g., a quencher, may be within no more than 5 to 20 nucleotides (e.g., within 7 or 10 nucleotides) of each other along the length of the oligonucleotide probe. In another aspect, the oligonucleotide probe includes a nucleic acid sequence that permits secondary structure formation. Such secondary structure formation may result in spatial proximity between the first and second fluorescent moiety. According to this method, the second fluorescent moiety on the oligonucleotide probe can be a quencher.

The present disclosure also provides for methods of preferentially and/or selectively detecting and/or quantifying a target RNA over DNA in a biological sample from an individual. These methods can be employed to detect the presence or absence of a target RNA in plasma, for use in blood screening and diagnostic testing. Additionally, the same test may be used by someone experienced in the art to assess urine and other sample types to detect and/or quantify target RNA nucleic acids preferentially and/or selectively over DNA nucleic acids. Such methods generally include performing at least one cycling step, which includes an amplifying step and a dye-binding step. Typically, the amplifying step includes contacting the sample with a plurality of pairs of oligonucleotide primers to produce one or more amplification products if a nucleic acid molecule is present in the sample, and the dye-binding step includes contacting the amplification product with a double-stranded DNA binding dye. Such methods also include detecting the presence or absence of binding of the double-stranded DNA binding dye into the amplification product, wherein the presence of binding is indicative of the presence of the target RNA nucleic acid in the sample, and wherein the absence of binding is indicative of the absence of the target RNA nucleic acid in the sample. A representative double-stranded DNA binding dye is ethidium bromide. Other nucleic acid-binding dyes include DAPI, Hoechst dyes, PicoGreen®, RiboGreen®, OliGreen®, and cyanine dyes such as YO-YO® and SYBR® Green. In addition, such methods also can include determining the melting temperature between the amplification product and the double-stranded DNA binding dye, wherein the melting temperature confirms the presence or absence of the target RNA nucleic acid.

In a further embodiment, a kit for preferentially and/or selectively detecting and/or quantitating one or more target RNA nucleic acids is provided. The kit can include one or more sets of oligonucleotide primers specific for amplification of the gene target; and one or more detectable oligonucleotide probes specific for detection of the amplification products, as well as one or more polymerases, and dNTPs (including dATP, dCTP, dTTP, dGTP, and dITP). In one aspect, the kit can include oligonucleotide probes already labeled with donor and corresponding acceptor moieties, e.g., another fluorescent moiety or a dark quencher, or can include fluorophoric moieties for labeling the oligonucleotide probes. The kit can also include nucleoside triphosphates, nucleic acid polymerase, and buffers necessary for the function of the nucleic acid polymerase. The kit can also include a package insert and instructions for using the oligonucleotide primers, oligonucleotide probes, and fluorophoric moieties to preferentially and/or selectively detect and/or quantitate a target RNA in a sample.

Unless otherwise defined, all technical and scientific terms used herein have the same meaning as commonly understood by one of ordinary skill in the art to which this invention belongs. Although methods and materials similar or equivalent to those described herein can be used in the practice or testing of the present subject matter, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE FIGURES

FIG. 1 shows the molecular difference between dITP and dGTP.

FIG. 2A shows an amplification curve and FIG. 2B shows a melting curve. These figures show that the Tm of the HBV Region 7 amplicon lowers as the dITP concentration increases. These experiments are described in Example 2.

FIGS. 3A-3C shows PCR growth curves showing that the master mix with 200 M dITP recovers performance at lower denaturation temperatures. These experiments are described in Example 3.

FIGS. 4A-4C shows PCR growth curves and melting curve showing that the 200 M dITP significantly demonstrates improved performance at lower denaturation temperatures. These experiments are described in Example 3.

FIGS. 5A-5C shows PCR growth curves and melting curve showing that the formulation with 200 μM dITP generates improved performance at lower denaturation temperatures. These experiments are described in Example 3.

FIGS. 6A and 6B shows a PCR growth curve showing the HBV Insert 3 (FIG. 6A) and GIC transcripts (FIG. 6B) were duplexed with TaqMan probes and tested on a denaturation temperature gradient. The formulation with 200 μM dITP generated improved performance especially at lower denaturation temperatures. These experiments are described in Example 4.

FIGS. 7A-7F show PCR growth curves from six different clinical plasma samples, showing reactions with dITP demonstrated selective amplification of RNA versus DNA when compared to reactions without dITP. These experiments are described in Example 5.

FIGS. 8A-8D show PCR growth curves, under various experimental conditions, showing that in the Cy5.5 channel, GIC minus dITP, minus RT hold reactions generate amplification curves which is unexpected. RT activity may be still present in these reactions even in the absence of the RT hold. This phenomenon is not observed in the plus dITP, minus RT hold reactions. These experiments are described in Example 5.

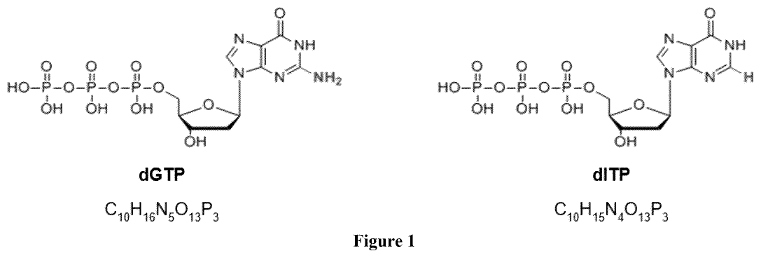

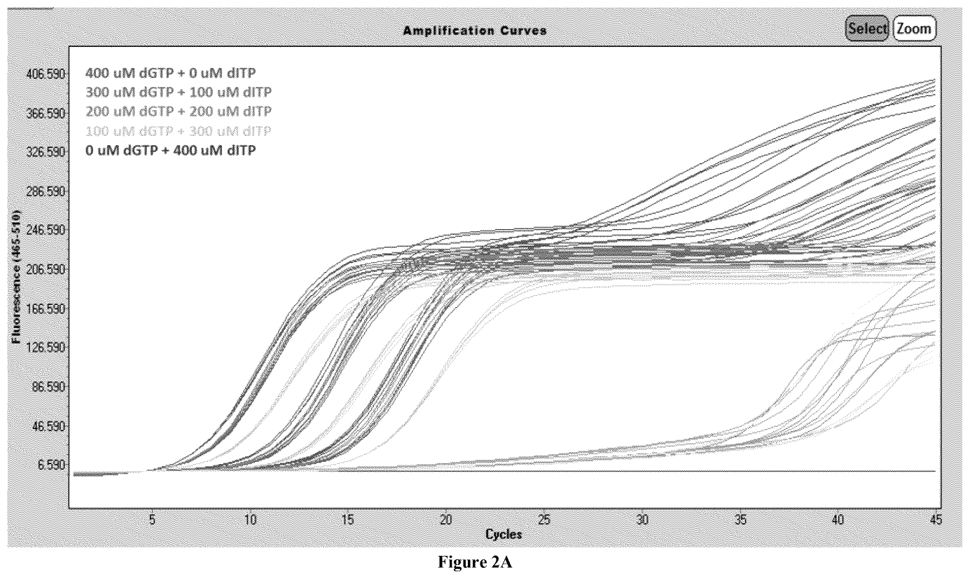

### DETAILED DESCRIPTION OF THE INVENTION

Methods, kits, and reaction mixtures for the preferential and/or selective detection and/or quantitation of a target RNA (over DNA) is described herein. In particular, polymerase(s), dNTPs (including dATP, dTTP, dCTP, dGTP, and dITP), primers, and probes for preferentially and/or selectively detecting and/or quantitating target RNA are provided, as are articles of manufacture or kits containing such reagents. Additionally, this technology may be employed for blood screening as well as for prognosis.

As used herein, the term “amplifying” refers to the process of synthesizing nucleic acid molecules that are complementary to one or both strands of a template nucleic acid molecule (e.g., nucleic acid molecules, such as RNA from the HBV genome). Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., Platinum® Taq) and an appropriate buffer and/or co-factors for optimal activity of the polymerase enzyme (e.g., MgCl₂ and/or KCl).

The term “primer” as used herein is known to those skilled in the art and refers to oligomeric compounds, primarily to oligonucleotides but also to modified oligonucleotides that are able to “prime” DNA synthesis by a template-dependent DNA polymerase, i.e., the 3′-end of the, e.g., oligonucleotide provides a free 3′-OH group where further “nucleotides” may be attached by a template-dependent DNA polymerase establishing 3′ to 5′ phosphodiester linkage whereby deoxynucleoside triphosphates are used and whereby pyrophosphate is released.

The term “hybridizing” refers to the annealing of one or more probes to an amplification product. “Hybridization conditions” typically include a temperature that is below the melting temperature of the probes but that avoids non-specific hybridization of the probes.

The term “5′ to 3′ nuclease activity” refers to an activity of a nucleic acid polymerase, typically associated with the nucleic acid strand synthesis, whereby nucleotides are removed from the 5′ end of nucleic acid strand.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished, if necessary.

The term “complement thereof” refers to nucleic acid that is both the same length as, and exactly complementary to, a given nucleic acid.

The term “extension” or “elongation” when used with respect to nucleic acids refers to when additional nucleotides (or other analogous molecules) are incorporated into the nucleic acids. For example, a nucleic acid is optionally extended by a nucleotide incorporating biocatalyst, such as a polymerase that typically adds nucleotides at the 3′ terminal end of a nucleic acid.

The terms “identical” or percent “identity” in the context of two or more nucleic acid sequences, refer to two or more sequences or subsequences that are the same or have a specified percentage of nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated herein by reference.

A “modified nucleotide” in the context of an oligonucleotide refers to an alteration in which at least one nucleotide of the oligonucleotide sequence is replaced by a different nucleotide that provides a desired property to the oligonucleotide. Exemplary modified nucleotides that can be substituted in the oligonucleotides described herein include, e.g., a t-butyl benzyl, a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxy-xanthosine, a pyrazolopyrimidine analog, a pseudo-dU, a nitro pyrrole, a nitro indole, 2′-0-methyl ribo-U, 2′-0-methyl ribo-C, an N4-ethyl-dC, an N6-methyl-dA, a 5-propynyl dU, a 5-propynyl dC, 7-deaza-deoxyguanosine (deaza G (u-deaza)) and the like. Many other modified nucleotides that can be substituted in the oligonucleotides are referred to herein or are otherwise known in the art. In certain embodiments, modified nucleotide substitutions modify melting temperatures (Tₘ) of the oligonucleotides relative to the melting temperatures of corresponding unmodified oligonucleotides. To further illustrate, certain modified nucleotide substitutions can reduce non-specific nucleic acid amplification (e.g., minimize primer dimer formation or the like), increase the yield of an intended target amplicon, and/or the like in some embodiments. Examples of these types of nucleic acid modifications are described in, e.g., U.S. Pat. No. 6,001,611, which is incorporated herein by reference. Other modified nucleotide substitutions may alter the stability of the oligonucleotide, or provide other desirable features.

The term “probe” refers to synthetically or biologically produced nucleic acids (DNA or RNA), which by design or selection, contain specific nucleotide sequences that allow them to hybridize under defined predetermined stringencies specifically (i.e., preferentially and/or selectively) to “target nucleic acids”, in the present case to an RNA (target) nucleic acid. A “probe” can be referred to as a “detection probe” meaning that it detects the target nucleic acid.

In some embodiments, the described probes can be labeled with at least one fluorescent label. In one embodiment, the probes can be labeled with a donor fluorescent moiety, e.g., a fluorescent dye, and a corresponding acceptor moiety, e.g., a quencher.

Designing oligonucleotides to be used as probes can be performed in a manner similar to the design of primers. Embodiments may use a single probe or a pair of probes for detection of the amplification product. Depending on the embodiment, the probe(s) use may comprise at least one label and/or at least one quencher moiety. As with the primers, the probes usually have similar melting temperatures, and the length of each probe must be sufficient for sequence-specific hybridization to occur but not so long that fidelity is reduced during synthesis. Oligonucleotide probes are generally 15 to 40 (e.g., 16, 18, 20, 21, 22, 23, 24, or 25) nucleotides in length.

### Polymerase Chain Reaction (PCR)

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in some embodiments include oligonucleotides capable of acting as points of initiation of nucleic acid synthesis within the described RNA target nucleic acid sequences. A primer can be purified from a restriction digest by conventional methods, or it can be produced synthetically. The primer is preferably single-stranded for maximum efficiency in amplification, but the primer can be double-stranded. Double-stranded primers are first denatured, i.e., treated to separate the strands. One method of denaturing double stranded nucleic acids is by heating.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 sec to 4 min (e.g., 1 min to 2 min 30 sec, or 1.5 min).

If the double-stranded template nucleic acid is denatured by heat, the reaction mixture is allowed to cool to a temperature that promotes annealing of each primer to its target sequence. The temperature for annealing is usually from about 35° C. to about 65° C. (e.g., about 40° C. to about 60° C.; about 45° C. to about 50° C.). Annealing times can be from about 10 sec to about 1 min (e.g., about 20 sec to about 50 sec; about 30 sec to about 40 sec). The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; about 60° C.). Extension times can be from about 10 sec to about 5 min (e.g., about 30 sec to about 4 min; about 1 min to about 3 min; about 1 min 30 sec to about 2 min).

PCR assays can employ nucleic acid such as RNA or DNA (cDNA). The template nucleic acid need not be purified; it may be a minor fraction of a complex mixture, such as target RNA contained in human cells. Target RNA nucleic acid molecules may be extracted from a biological sample by routine techniques such as those described in Diagnostic Molecular Microbiology: Principles and Applications (Persing et al. (eds), 1993, American Society for Microbiology, Washington D.C.). Nucleic acids can be obtained from any number of sources, such as plasmids, or natural sources including bacteria, yeast, viruses, organelles, or higher organisms such as plants or animals.

The oligonucleotide primers are combined with PCR reagents under reaction conditions that induce primer extension. Such PCR reagents include, but are not limited to, one or more polymerases, and dNTPs (including dATP, dTTP, dCTP, dGTP, and dITP). For example, chain extension reactions generally include 50 mM KCl, 10 mM Tris-HCl (pH 8.3), 15 mM MgCl₂, 0.001% (w/v) gelatin, 0.5-1.0 μg denatured template DNA, 50 pmoles of each oligonucleotide primer, 2.5 U of Taq polymerase, and 10% DMSO). The reactions usually contain 150 to 320 μM each of dATP, dCTP, dTTP, dGTP, and dITP, or one or more analogs thereof.

The newly-synthesized strands form a double-stranded molecule that can be used in the succeeding steps of the reaction. The steps of strand separation, annealing, and elongation can be repeated as often as needed to produce the desired quantity of amplification products corresponding to the target RNA nucleic acid molecules. The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times.

### Fluorescence Resonance Energy Transfer (FRET)

FRET technology (see, for example, U.S. Pat. Nos. 4,996,143, 5,565,322, 5,849,489, and 6,162,603) is based on a concept that when a donor fluorescent moiety and a corresponding acceptor fluorescent moiety are positioned within a certain distance of each other, energy transfer takes place between the two fluorescent moieties that can be visualized or otherwise detected and/or quantitated. The donor typically transfers the energy to the acceptor when the donor is excited by light radiation with a suitable wavelength. The acceptor typically re-emits the transferred energy in the form of light radiation with a different wavelength. In certain systems, non-fluorescent energy can be transferred between donor and acceptor moieties, by way of biomolecules that include substantially non-fluorescent donor moieties (see, for example, U.S. Pat. No. 7,741,467).

In one example, an oligonucleotide probe can contain a donor fluorescent moiety or dye (e.g., HEX or FAM) and a corresponding quencher (e.g., BlackHole Quencher™ (BHQ) (such as BHQ-2)), which may or not be fluorescent, and which dissipates the transferred energy in a form other than light. When the probe is intact, energy transfer typically occurs between the donor and acceptor moieties such that fluorescent emission from the donor fluorescent moiety is quenched the acceptor moiety. During an extension step of a polymerase chain reaction, a probe bound to an amplification product is cleaved by the 5′ to 3′ nuclease activity of, e.g., a Taq Polymerase such that the fluorescent emission of the donor fluorescent moiety is no longer quenched. Exemplary probes for this purpose are described in, e.g., U.S. Pat. Nos. 5,210,015, 5,994,056, and 6,171,785. Commonly used donor-acceptor pairs include the FAM-TAMRA pair. Commonly used quenchers are DABCYL and TAMRA. Commonly used dark quenchers include BlackHole Quencher™ (BHQ) (such as BHQ2), (Biosearch Technologies, Inc., Novato, Cal.), Iowa Black™ (Integrated DNA Tech., Inc., Coralville, Iowa), BlackBerry™ Quencher 650 (BBQ-650), (Berry & Assoc., Dexter, Mich.).

In another example, two oligonucleotide probes, each containing a fluorescent moiety, can hybridize to an amplification product at particular positions determined by the complementarity of the oligonucleotide probes to the target RNA target nucleic acid sequence. Upon hybridization of the oligonucleotide probes to the amplification product nucleic acid at the appropriate positions, a FRET signal is generated. Hybridization temperatures can range from about 35° C. to about 65° C. for about 10 sec to about 1 min.

Fluorescent analysis can be carried out using, for example, a photon counting epifluorescent microscope system (containing the appropriate dichroic mirror and filters for monitoring fluorescent emission at the particular range), a photon counting photomultiplier system, or a fluorimeter. Excitation to initiate energy transfer, or to allow direct detection of a fluorophore, can be carried out with an argon ion laser, a high intensity mercury (Hg) arc lamp, a xenon lamp, a fiber optic light source, or other high intensity light source appropriately filtered for excitation in the desired range.

As used herein with respect to donor and corresponding acceptor moieties “corresponding” refers to an acceptor fluorescent moiety or a dark quencher having an absorbance spectrum that overlaps the emission spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced therebetween.

Fluorescent donor and corresponding acceptor moieties are generally chosen for (a) high efficiency Foerster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, helium-cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC Red 640, LC Red 705, Cy5, Cy5.5, Lissamine rhodamine B sulfonyl chloride, tetramethyl rhodamine isothiocyanate, rhodamine×isothiocyanate, erythrosine isothiocyanate, fluorescein, diethylenetriamine pentaacetate, or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and acceptor fluorescent moieties. The length of a linker arm can be the distance in Angstroms (Å) from the nucleotide base to the fluorescent moiety. In general, a linker arm is from about 10 Å to about 25 Å. The linker arm may be of the kind described in WO 84/03285. WO 84/03285 also discloses methods for attaching linker arms to a particular nucleotide base, and also for attaching fluorescent moieties to a linker arm.

An acceptor fluorescent moiety, such as an LC Red 640, can be combined with an oligonucleotide that contains an amino linker (e.g., C6-amino phosphoramidites available from ABI (Foster City, Calif.) or Glen Research (Sterling, VA)) to produce, for example, LC Red 640-labeled oligonucleotide. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as CX-fluorescein-CPG from BioGenex (San Ramon, Calif)), or 3′-amino-CPGs that require coupling of a fluorescein-NHS-ester after oligonucleotide synthesis.

Detection and/or Quantitation of the Target RNA Amplified Product (Amplicon)

The present disclosure provides methods for preferentially and/or selectively detecting and/or quantitating target RNA (over DNA) in a biological or non-biological sample. Methods provided avoid problems of sample contamination, false negatives, and false positives. The methods include performing at least one cycling step that includes preferentially and/or selectively amplifying a portion of target RNA from a sample using one or more pairs of target RNA primers, and a FRET detecting step. Multiple cycling steps are performed, preferably in a thermocycler. Methods can be performed using the target RNA primers and probes to preferentially and/or selectively detect and/or quantitate the target RNA (over DNA), wherein the presence of target RNA, and the detection of target RNA indicates the presence of target RNA in the sample.

As described herein, amplification products can be detected using labeled hybridization probes that take advantage of FRET technology. One FRET format utilizes TaqMan® technology to detect the presence or absence of an amplification product, and hence, the presence or absence of target RNA. TaqMan® technology utilizes one single-stranded hybridization probe labeled with, e.g., one fluorescent moiety or dye (e.g., HEX or FAM) and one quencher (e.g., BHQ-2), which may or may not be fluorescent. When a first fluorescent moiety is excited with light of a suitable wavelength, the absorbed energy is transferred to a second fluorescent moiety or a dark quencher according to the principles of FRET. The second moiety is generally a quencher molecule. During the annealing step of the PCR reaction, the labeled hybridization probe binds to the target DNA (i.e., the amplification product) and is degraded by the 5′ to 3′ nuclease activity of, e.g., the Taq Polymerase during the subsequent elongation phase. As a result, the fluorescent moiety and the quencher moiety become spatially separated from one another. As a consequence, upon excitation of the first fluorescent moiety in the absence of the quencher, the fluorescence emission from the first fluorescent moiety can be detected. By way of example, an ABI PRISM® 7700 Sequence Detection System (Applied Biosystems) uses TaqMan® technology, and is suitable for performing the methods described herein for preferentially and/or selectively detecting and/or quantiating target RNA (over DNA) in the sample.

Molecular beacons in conjunction with FRET can also be used to detect the presence of an amplification product using the real-time PCR methods. Molecular beacon technology uses a hybridization probe labeled with a first fluorescent moiety and a second fluorescent moiety. The second fluorescent moiety is generally a quencher, and the fluorescent labels are typically located at each end of the probe. Molecular beacon technology uses a probe oligonucleotide having sequences that permit secondary structure formation (e.g., a hairpin). As a result of secondary structure formation within the probe, both fluorescent moieties are in spatial proximity when the probe is in solution. After hybridization to the target nucleic acids (i.e., amplification products), the secondary structure of the probe is disrupted and the fluorescent moieties become separated from one another such that after excitation with light of a suitable wavelength, the emission of the first fluorescent moiety can be detected.

Another common format of FRET technology utilizes two hybridization probes. Each probe can be labeled with a different fluorescent moiety and are generally designed to hybridize in close proximity to each other in a target DNA molecule (e.g., an amplification product). A donor fluorescent moiety, for example, fluorescein, is excited at 470 nm by the light source of the LightCycler® Instrument. During FRET, the fluorescein transfers its energy to an acceptor fluorescent moiety such as LightCycler®-Red 640 (LC Red 640) or LightCycler®-Red 705 (LC Red 705). The acceptor fluorescent moiety then emits light of a longer wavelength, which is detected by the optical detection system of the LightCycler® instrument. Efficient FRET can only take place when the fluorescent moieties are in direct local proximity and when the emission spectrum of the donor fluorescent moiety overlaps with the absorption spectrum of the acceptor fluorescent moiety. The intensity of the emitted signal can be correlated with the number of original target DNA/RNA molecules (e.g., the number of target RNA molecules). If amplification of target RNA occurs and an amplification product is produced, the step of hybridizing results in a detectable signal based upon FRET between the members of the pair of probes.

Generally, the presence of FRET indicates the presence of the target RNA in the sample, and the absence of FRET indicates the absence of the target RNA in the sample. Inadequate specimen collection, transportation delays, inappropriate transportation conditions, or use of certain collection swabs (calcium alginate or aluminum shaft) are all conditions that can affect the success and/or accuracy of a test result, however.

Representative biological samples that can be used in practicing the methods include, but are not limited to whole blood, respiratory specimens, urine, fecal specimens, blood specimens, plasma, dermal swabs, nasal swabs, wound swabs, blood cultures, skin, and soft tissue infections. Collection and storage methods of biological samples are known to those of skill in the art. Biological samples can be processed (e.g., by nucleic acid extraction methods and/or kits known in the art) to release nucleic acids (such as the target RNA) or in some cases, the biological sample can be contacted directly with the PCR reaction components and the appropriate oligonucleotides. In some instances, the biological sample is whole blood. When whole blood is typically collected, it is often collected in vessels containing anticoagulants, such as heparin, citrate, or EDTA, which enables the whole blood to be stored at suitable temperatures. However, under such conditions, the nucleic acids within the whole blood undergo considerable amount of degradation. Therefore, it may be advantageous to collect the blood in a reagent that will lyse, denature, and stabilize whole blood components, including nucleic acids, such as a nucleic acid-stabilizing solution. In such cases, the nucleic acids can be better preserved and stabilized for subsequent isolation and analysis, such as by nucleic acid test, such as PCR. Such nucleic acid-stabilizing solution are well known in the art, including, but not limited to, Cobas® PCR media, which contains 4.2 M guanadinium salt (GuHCl) and 50 mM Tris, at a pH of 7.5.

The sample can be collected by any method or device designed to adequately hold and store the sample prior to analysis. Such methods and devices are well known in the art. In the case that the sample is a biological sample, such as whole blood, the method or device may include a blood collection vessel. Such a blood collection vessel is well known in the art, and may include, for example, a blood collection tube. In many cases, it may be advantageous to use a blood collection tube wherein the blood collection vessel is under pressure in the space intended for sample uptake, such as a blood vessel with an evacuated chamber, such as a vacutainer blood collection tube. Such blood collection tubes with an evacuated chamber, such as a vacutainer blood collection tube are well known in the art. It may further be advantageous to collect the blood in a blood collection vessel, with or without an evacuated chamber, that contains within it, a solution that will lyse, denature, and stabilize whole blood components, including nucleic acids, such as a nucleic acid-stabilizing solution, such that the whole blood being drawn immediately contacts the nucleic acid-stabilizing solution in the blood collection vessel.

Melting curve analysis is an additional step that can be included in a cycling profile. Melting curve analysis is based on the fact that DNA/RNA melts at a characteristic temperature called the melting temperature (Tₘ), which is defined as the temperature at which half of the nucleic acid duplexes have separated into single strands. The melting temperature of a DNA/RNA depends primarily upon its nucleotide composition. Thus, nucleic acid molecules rich in G and C nucleotides have a higher Tm than those having an abundance of A and T nucleotides. By detecting the temperature at which signal is lost, the melting temperature of probes can be determined. Similarly, by detecting the temperature at which signal is generated, the annealing temperature of probes can be determined. The melting temperature(s) of the target RNA probes from the target RNA amplification products can confirm the presence or absence of target RNA in the sample.

Within each thermocycler run, control samples can be cycled as well. Positive control samples can amplify target nucleic acid control template (other than described amplification products of target genes) using, for example, control primers and control probes. Positive control samples can also amplify, for example, a plasmid construct containing the target nucleic acid molecules. Such a plasmid control can be amplified internally (e.g., within the sample) or in a separate sample run side-by-side with the patients' samples using the same primers and probe as used for detection of the intended target. Such controls are indicators of the success or failure of the amplification, hybridization, and/or FRET reaction. Each thermocycler run can also include a negative control that, for example, lacks target template DNA. Negative control can measure contamination. This ensures that the system and reagents would not give rise to a false positive signal. Therefore, control reactions can readily determine, for example, the ability of primers to anneal with sequence-specificity and to initiate elongation, as well as the ability of probes to hybridize with sequence-specificity and for FRET to occur.

In an embodiment, the methods include steps to avoid contamination. For example, an enzymatic method utilizing uracil-DNA glycosylase is described in U.S. Pat. Nos. 5,035,996, 5,683,896 and 5,945,313 to reduce or eliminate contamination between one thermocycler run and the next. Conventional PCR methods in conjunction with FRET technology can be used to practice the methods. In one embodiment, a LightCycler® instrument is used. The following patent applications describe real-time PCR as used in the LightCycler® technology: WO 97/46707, WO 97/46714, and WO 97/46712.

The LightCycler® can be operated using a PC workstation and can utilize a Windows NT operating system. Signals from the samples are obtained as the machine positions the capillaries sequentially over the optical unit. The software can display the fluorescence signals in real-time immediately after each measurement. Fluorescent acquisition time is 10-100 milliseconds (msec). After each cycling step, a quantitative display of fluorescence vs. cycle number can be continually updated for all samples. The data generated can be stored for further analysis.

As an alternative to FRET, an amplification product can be detected using a double-stranded DNA binding dye such as a fluorescent DNA binding dye (e.g., SYBR® Green or SYBR® Gold (Molecular Probes)). Upon interaction with the double-stranded nucleic acid, such fluorescent DNA binding dyes emit a fluorescence signal after excitation with light at a suitable wavelength. A double-stranded DNA binding dye such as a nucleic acid intercalating dye also can be used. When double-stranded DNA binding dyes are used, a melting curve analysis is usually performed for confirmation of the presence of the amplification product.

One of skill in the art would appreciate that other nucleic acid- or signal-amplification methods may also be employed. Examples of such methods include, without limitation, branched DNA signal amplification, loop-mediated isothermal amplification (LAMP), nucleic acid sequence-based amplification (NASBA), self-sustained sequence replication (3SR), strand displacement amplification (SDA), or smart amplification process version 2 (SMAP 2).

It is understood that the embodiments of the present disclosure are not limited by the configuration of one or more commercially available instruments.

### Articles of Manufacture/Kits

Embodiments of the present disclosure further provide for articles of manufacture or kits to preferentially and/or selectively detect and/or quantitate target RNA (over DNA). An article of manufacture can include primers and probes used to preferentially and/or selectively detect and/or quantitate the target RNA, together with suitable packaging materials, including dNTPs (including dATP, dCTP, dTTP, dGTP, and dITP). Representative primers and probes for the preferential and/or selective detection and/or quantitation of target RNA (over DNA) are capable of hybridizing to target RNA molecules. In addition, the kits may also include suitably packaged reagents and materials needed for DNA immobilization, hybridization, and detection, such solid supports, buffers, enzymes, and DNA standards. Methods of designing primers and probes are disclosed herein, and representative examples of primers and probes that preferentially and/or selectively amplify and hybridize to RNA target molecules are provided.

Articles of manufacture can also include one or more fluorescent moieties for labeling the probes or, alternatively, the probes supplied with the kit can be labeled. For example, an article of manufacture may include a donor and/or an acceptor fluorescent moiety for labeling the target RNA probes. Examples of suitable FRET donor fluorescent moieties and corresponding acceptor fluorescent moieties are provided above.

Articles of manufacture can also contain a package insert or package label having instructions thereon for using the target RNA primers and probes to detect target RNA in a sample. Articles of manufacture may additionally include reagents for carrying out the methods disclosed herein (e.g., buffers, polymerase enzymes, co-factors, or agents to prevent contamination). Such reagents may be specific for one of the commercially available instruments described herein.

Embodiments of the present disclosure also provide for a set of primers and one or more detectable probes for the preferentially and/or selectively detection and/or quantitation of target RNA in a sample.

Embodiments of the present disclosure will be further described in the following examples, which do not limit the scope of the invention described in the claims.

### EXAMPLES

The following examples and figures are provided to aid the understanding of the subject matter, the true scope of which is set forth in the appended claims. It is understood that modifications can be made in the procedures set forth without departing from the spirit of the invention. In all of the following Examples, the polymerase employed was the Z05D polymerase, which is a D580G mutant of the Z05 polymerase, described in, for example, U.S. Pat. Nos. 8,962,293, 9,102,924, and 9,738,876.

### Example 1: Approach of Testing the Effect of dITP in Hepatitis B Virus (HBV) Assay

HBV is a partially double-stranded DNA virus which packages a single-stranded RNA pre-genomic (pgRNA) that is reverse transcribed. Current treatments for chronic HBV infection include nucleos(t)ide analogues (NA) which disrupts reverse transcription of pgRNA to suppress DNA synthesis. It has been indicated that HBV pgRNA is a surrogate for cccDNA (covalently closed circular DNA) and is detected in plasma even when HBV DNA production is suppressed. Due to severe side effects and long-term benefits seen in less than half of treated patients, patient monitoring for treatment cessation is critical. Current monitoring methods involve HBV DNA quantitation or HBV antigen detection, both of which are considered to have limitations. These markers may be inadequate to reflect cccDNA activity, since NA therapy does not directly eliminate the cccDNA reservoir. Therefore, there is a possibility that discontinuing NA therapy can cause rebound in HBV infection. Since the continued presence of HBV RNA particles may indicate the active transcription of the cccDNA, HBV pgRNA may be a potential biomarker to identify long-term viral suppressed patients who may be candidates for treatment cessation.

Thus, an HBV pgRNA quantitative assay would be useful for monitoring patient treatment cessation. One of the critical challenges in developing an HBV pgRNA assay would, however, be in distinguishing between HBV RNA versus viral DNA amplification. Exclusion of HBV DNA is difficult to achieve due to the limited number of sequence and structural differences to HBV pgRNA which can be targeted. Therefore, the development of an HBV quantitative assay provided a platform to assess the utility of dITPs to ensure RNA selectivity in a useful way.

To assess the effect of dITP, an HBV assay was developed in HBV Region 7. The HBV Region 7 amplicon is referred to as Insert 3. In the interest of avoiding divalent metal ion adjustment (Mn(OAc)₂), the total dNTP concentration of 2.0 mM remained constant and only ratios of dGTP and dITP were varied to determine the optimal concentrations for the HBV Region 7 and Generic Internal Control (GIC) assays. The GIC assay was evaluated along with the HBV Region 7 assay because the GIC assay detects a generic internal control that serves as a Full Process Control (FPC), an Internal Control (IC), and an Internal Quantitation Standard (IQS) in all of the Cobas® 6800/8800 assays. The optimal dGTP:dITP ratio depends on the GC-content of the primers, probe, and amplicon. Determining the ideal denaturation temperature for selective RNA amplification is dependent on the amounts of dIMP that is incorporated and the length of the amplicon. It is noted that dIMP, a dITP derivative, is the monomer form of dITP that is incorporated into the DNA. The HBV Region 7 and the GIC assay sequences, Tₘ (° C.), and GC-content are listed in Tables 1 and 2, respectively, below.

TABLE 1
HBV Region 7 assay sequence
HBV Region 7 Assay

| | | | | | SEQ |
|---|---|---|---|---|---|
| | | Tm | GC | nMER | ID |
| Name | Sequence | ° C. | Content | (bp) | NO: |
| REGION7_ | ACTNCCGGAAACNACNGTNGTNAGJ | 63.3 | 0.40 | 25 | 1 |
| AF4BF1B_P | Modifications: | | | | |
| ANM | N: C-5 propyny1-dU | | | | |
| (Forward) | J: t-Butyl Benzyl-dA | | | | |
| REGION7_ | CCCGAGANTGAGANCTTMTGMGAK | 68.3 | 0.55 | 24 | 2 |
| AR3B_M2 | Modifications: | | | | |
| (Reverse) | M: 5-Methyl-dC | | | | |
| | N: C-5 propyny1-dU | | | | |
| | K: t-Butyl Benzyl-dC | | | | |
| REGION7_ | HCMNAGAAGQAAGAAMTCCCTCGCCTCGCAG | 78.6 | 0.59 | 35 | 3 |
| AP1ENDM | ACGP | | | | |
| (HEX-probe) | Modifications: | | | | |
| | H: HEX-Thr | | | | |
| | M: 5-Methyl-dC | | | | |
| | N: C-5 propyny1-dU | | | | |
| | Q: BHQ-2 | | | | |
| | P: Phosphate | | | | |
| Insert 3 | ACTTCCGGAAACTACTGTTGTTAGACGACGAG | 88.0 | 0.55 | 113 | 4 |
| Amplicon | GCAGGTCCCCTAGAAGAAGAACTCCCTCGCCT | | | | |
| | CGCAGACGAAGGTCTCAATCGCCGCGTCGCAG | | | | |
| | AAGATCTCAATCTCGGG | | | | |

TABLE 2
GIC assay sequence
GIC Assay

| | | | | | SEQ |
|---|---|---|---|---|---|
| | | Tm | GC | nMER | ID |
| Name | Sequence | ° C. | Content | (bp) | NO: |
| EFIC3PRIT | ACAACCGCGCCATACATGTCAAGAJ | 69.0 | 0.48 | 25 | 5 |
| BB | Modifications: | | | | |
| (Forward) | J: t-Butyl Benzyl-dA | | | | |
| EFIC3PRIR | GTCGGGCCGCTTATACAGTACCAJ | 68.0 | 0.54 | 24 | 6 |
| TBB | Modifications: | | | | |
| (Reverse | J: t-Butyl Benzyl-dA | | | | |
| GIC10RCY5 | BTGCGCGTCCCGQTTTTGATACTTCGTAACGGT | 76.0 | 0.54 | 36 | 7 |
| 5BQ11 | GCP | | | | |
| (Cy5.5 | Modifications: | | | | |
| probe) | B: Cy5.5 | | | | |
| | Q: BHQ-2 | | | | |
| | P: Phoshate | | | | |
| Amplicon | ACAACCGCGCCATACATGTCAAGAATGAAGTG | 83.0 | 0.51 | 60 | 8 |
| (pEF070) | GGCGAACGCTAGAAAACTGACGCCAGCA | | | | |

Initial experiments were executed with SYTO 9 intercalating dye to view the melting products and the effect of dITP on the amplicon Tₘ. The SYTO 9 experiments were performed on HBV Insert 3 transcript, HBV Insert 3 DNA gBlock (double-stranded DNA fragment that is identical to the HBV transcript sequence), and the GIC transcript. Subsequently, HBV and GIC duplex reactions with TaqMan probes were evaluated with various dGTP:dITP ratios across denaturation and annealing temperature gradients to determine the optimal dITP concentration and thermal cycling parameters. A performance assessment of the HBV Insert 3 transcript spiked with HBV Insert 3 DNA gBlock was executed to examine the amplification efficiency and specificity of the HBV RNA target in comparison to the HBV DNA gBlock. After finalizing the dITP concentration and thermal cycling temperatures, clinical HBV plasma samples of varying viral loads extracted by the Cobas® 6800/8800 were evaluated with the optimized dITP conditions.

### Example 2: Use of dITP Results in Reduction in Tₘ

Thus, the HBV Region 7 Assay was conducted using a dITP titration with SYTO 9. The effect of dITP on the HBV Insert 3 amplicon Tₘ was assessed utilizing SYTO 9 dye (1 M/reaction). The HBV Region 7 assay was tested with different ratios of dGTP:dITP, so as to optimize the assay. The final dNTP concentration in the master mix was 2.0 mM, which consists of 400 μM dATP, 400 μM dCTP, 800 μM dUTP, and 400 uM (dGTP+dITP). The following five formulations of varying dGTP and dITP ratios were tested on the backbone Cobas® 6800/8800 master mix, are shown in Table 3, below:

TABLE 3
Five formulations for varying dGTP and dITP ratios tested on
the backbone cobas ® 6800/8800 master mix.

400 μM dGTP + 0 μM dITP
300 μM dGTP + 100 μM dITP
200 μM dGTP + 200 μM dITP
100 μM dGTP + 300 μM dITP
0 μM dGTP + 400 μM dITP

A titration of HBV Insert 3 transcript was tested at 0.1, 0.01, and 0.001 ng/reaction. The generic Cobas® 6800/8800 thermal profile (which includes denaturation temperatures of 95° C. and 91° C.) was tested in the experiment, listed in Table 4, below.

TABLE 4
Generic cobas ® 6800/8800 thermal profile.
Generic cobas ® 6800/8800 Thermal Cycling Profile

| | Cy- | Temperature | Hold time | Acqui- | |
|---|---|---|---|---|---|
| Step | cles | (° C.) | (hh:mm:ss) | sition | Ramp |
| UNG | 1 | 50 | 0:02:00 | — | 4.4 |
| UNG Deactivation | | 94 | 0:00:05 | — | 4.4 |
| RT | | 55 | 0:02:00 | — | 2.2 |
| | | 60 | 0:06:00 | — | 4.4 |
| | | 65 | 0:04:00 | — | 4.4 |
| PCR | 5 | 95 | 0:00:05 | — | 4.4 |
| | | 55 | 0:00:30 | — | 2.2 |
| | 45 | 91 | 0:00:05 | — | 4.4 |
| | | 58 | 0:00:25 | Single | 2.2 |
| Cool-down | 1 | 40 | 0:02:00 | — | 2.2 |

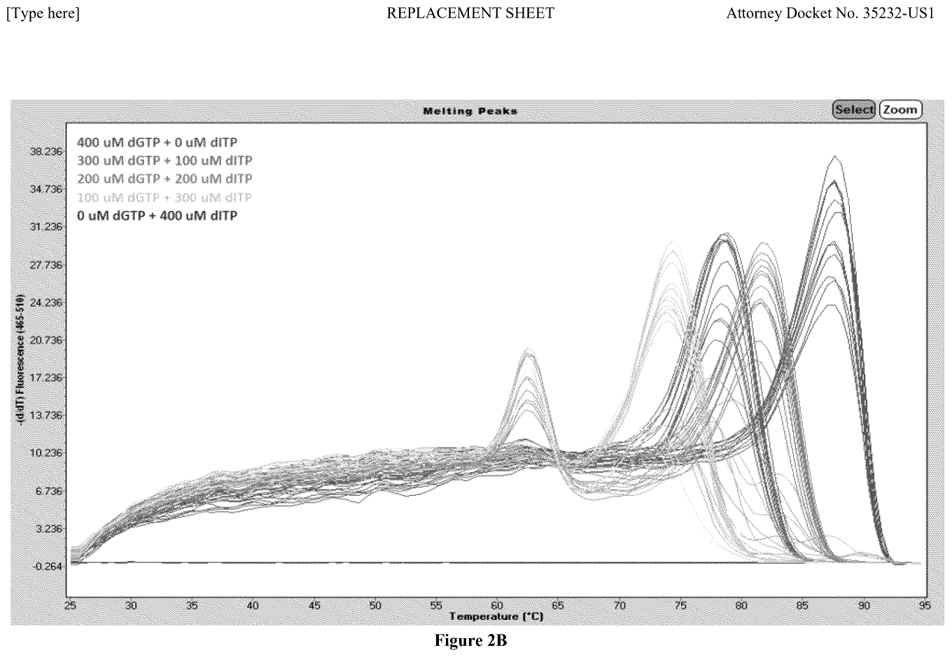

The results are shown in FIGS. 2A and 2B. The amplification curves are shown in FIG. 2A. The melting curves shown in FIG. 2B show that across all the formulations, amplification curves were generated except for the master mix in the absence of dGTP. This indicates that at least some dGTP is necessary in order to amplify the target under the thermal cycling conditions tested. The Tm of the amplicon reduces as the concentration of dITP in the reaction increases. The average amplicon Tm of the minus dITP reactions is 87.2° C. The amplicon Tm of the formulation with 300 M dITP was significantly reduced to 74.1° C. From this experiment, the effect of dITP on the HBV Insert 3 amplicon was demonstrated by the resulting 13° C. reduction in Tm. Thus, these studies demonstrate that as the concentration of dITP increases, the Tm of the amplicon decreases.

### Example 3: DITP Performance Evaluation on the HBV Region 7 and gBlock and Generic Internal Control (GIC) Transcripts

A gBlock double-stranded DNA fragment with an identical sequence to the HBV Region 7 transcript was evaluated. Since viral HBV RNA and HBV DNA have very similar sequences, evaluations of HBV gBlock and HBV transcript target pool was of interest to determine the HBV RNA amplification efficiency and specificity. Initial experiments with SYTO 9 dye were tested with the HBV Region 7 transcript, HBV Region 7 DNA gBlock and the GIC transcript to view the amplicon melting curves and Tₘ. Two master mix formulations containing 100 M and 200 M dITP were assessed with all three targets across a denaturation temperature gradient ranging from 79.0 to 87.0° C. on the Roche LightCycler© 96. The SYTO 9 dye was tested at 1 M and a melting step was included at the end of the thermal profile. All of the targets were tested in singleplex, because SYTO 9 intercalating dye was present in the reactions. The HBV Insert 3 transcript, gBlock, and the GICtranscript were tested at one concentration.

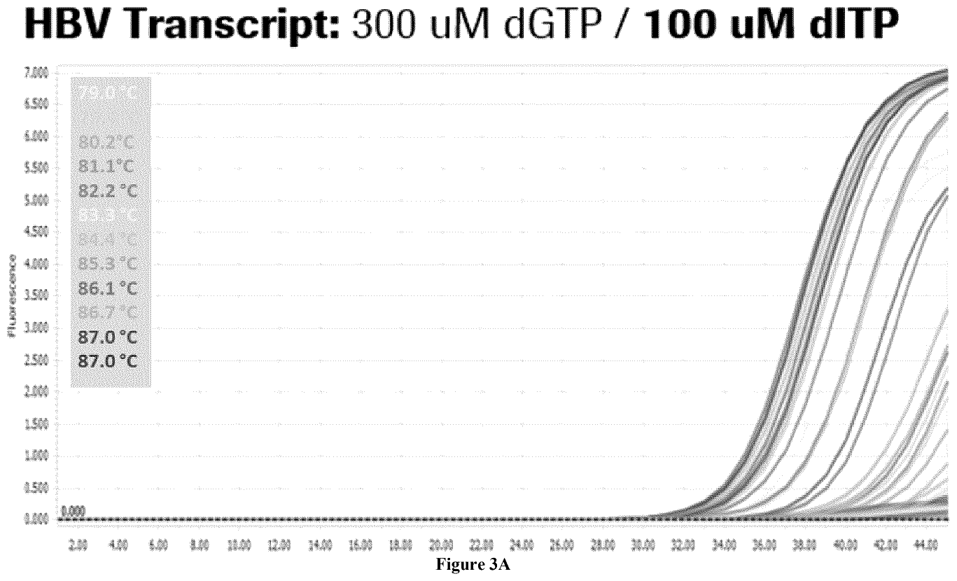

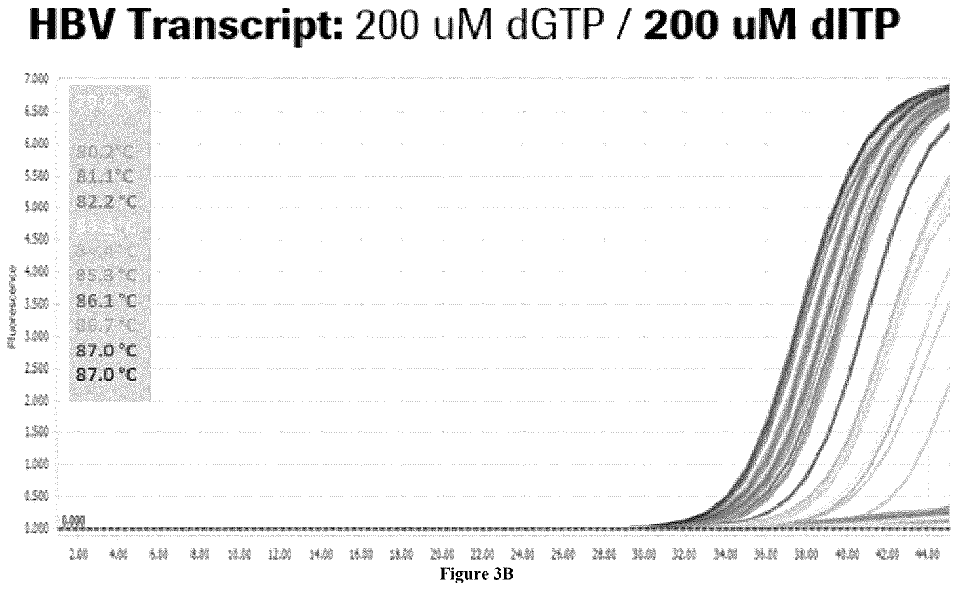

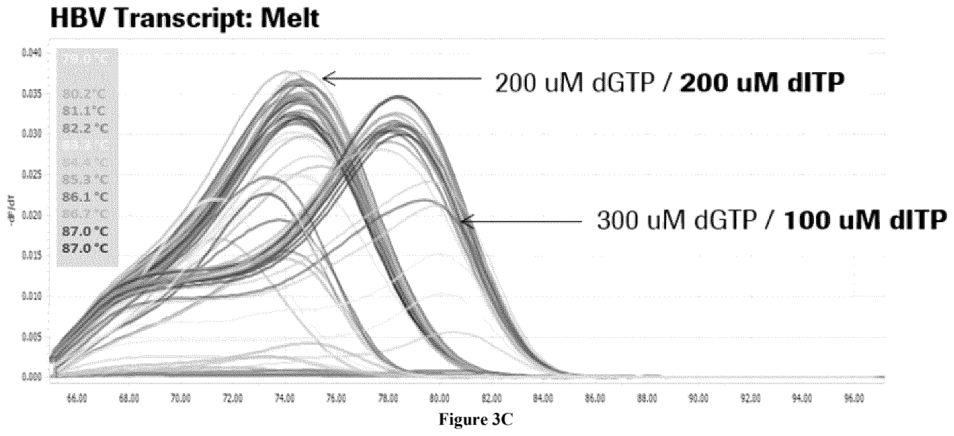

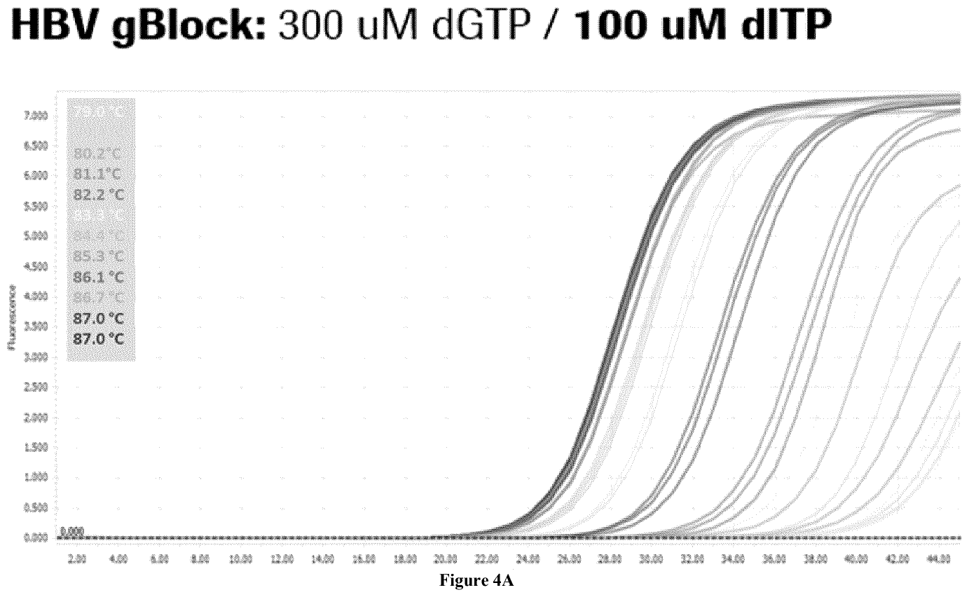

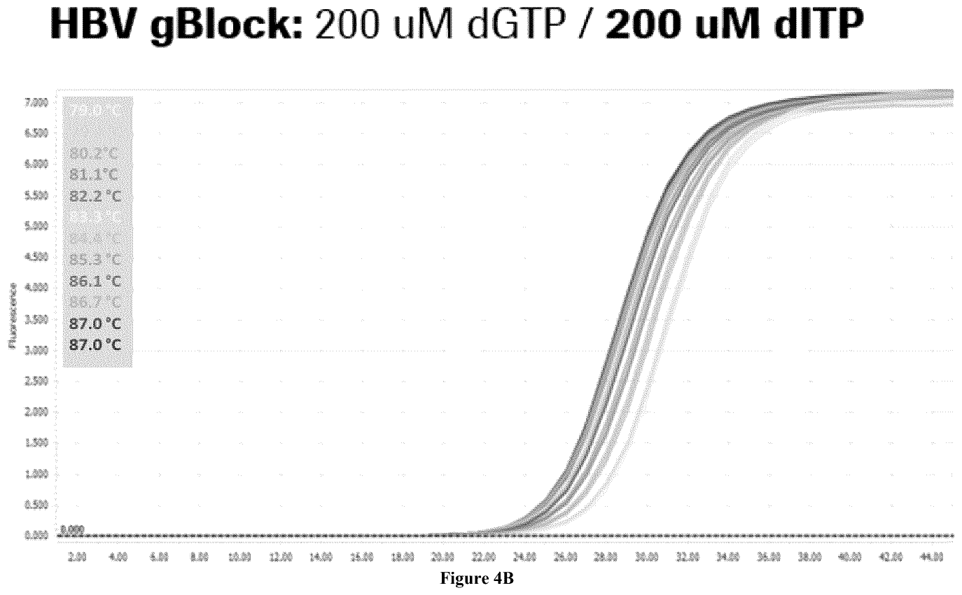

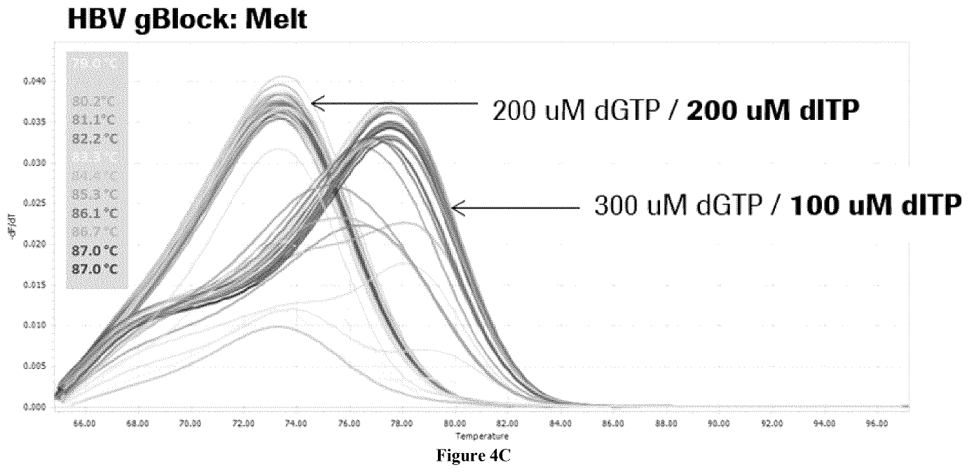

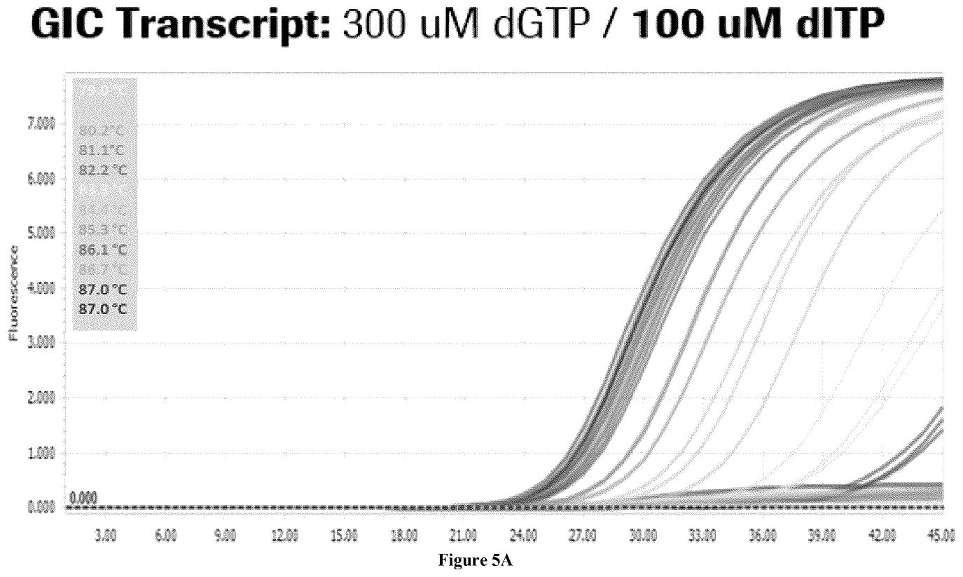

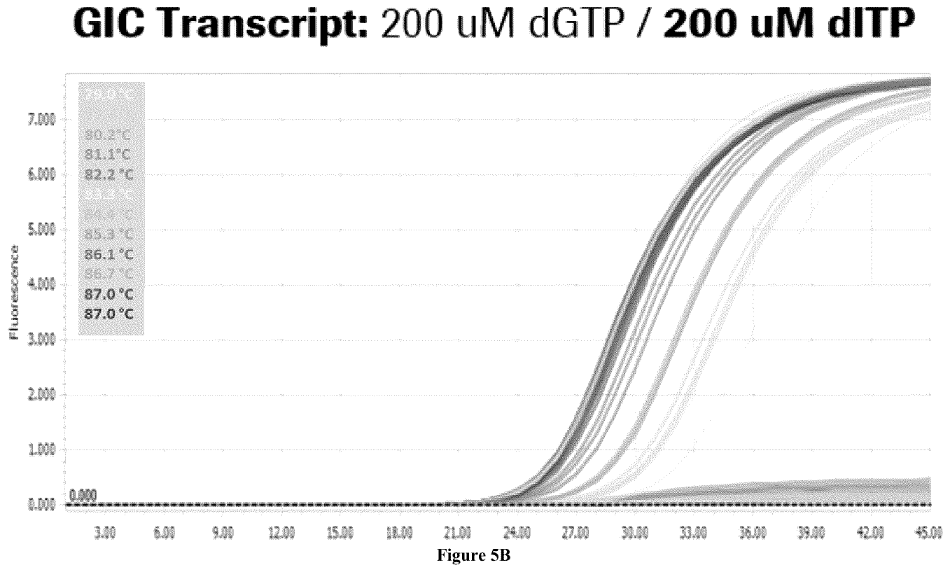

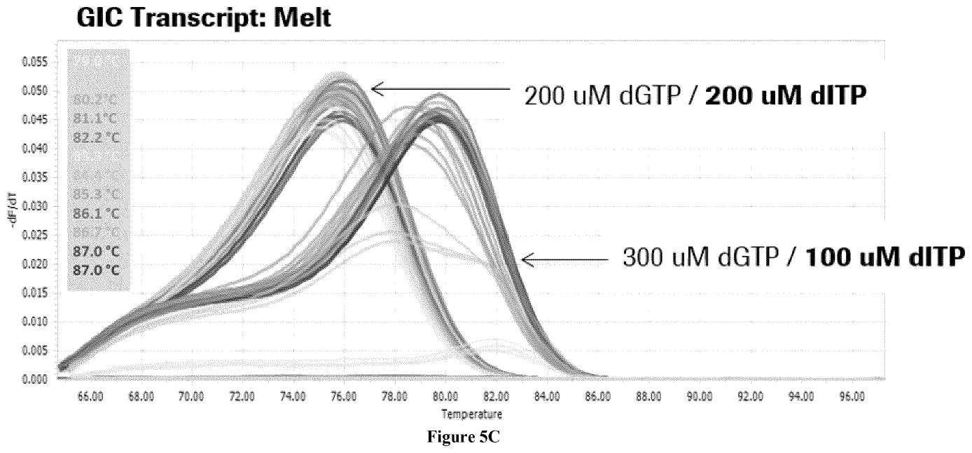

Results show that the master mix with 200 M dITP (and 200 M dGTP) (FIG. 3B) demonstrates considerably improved performance across all three targets at the lower denaturation temperatures in comparison to the master mix with just 100 M dITP (and 300 M dGTP) (FIG. 3A). The master mix with 200 μM dITP (and 200 μM dGTP) reliably amplified the HBV Region 7 transcript down to a denaturation temperature of 81.1° C., which is at least 10° C. lower than the denaturation temperature of the generic Cobas® 6800/8800 thermal profile, shown in Table 3, above, and as shown in FIGS. 3A-3C. As shown in FIGS. 4A-4C and 5A-5C, it can also amplify singleplex reactions of the HBV Region 7 gBlock, presumably due to the short length (318 bp), and reliably detect the GIC transcript down to a denaturation temperature around 79.0° C.

### Example 4: HBV Region 7 and GIC Duplexed Performance Assessment with dITP

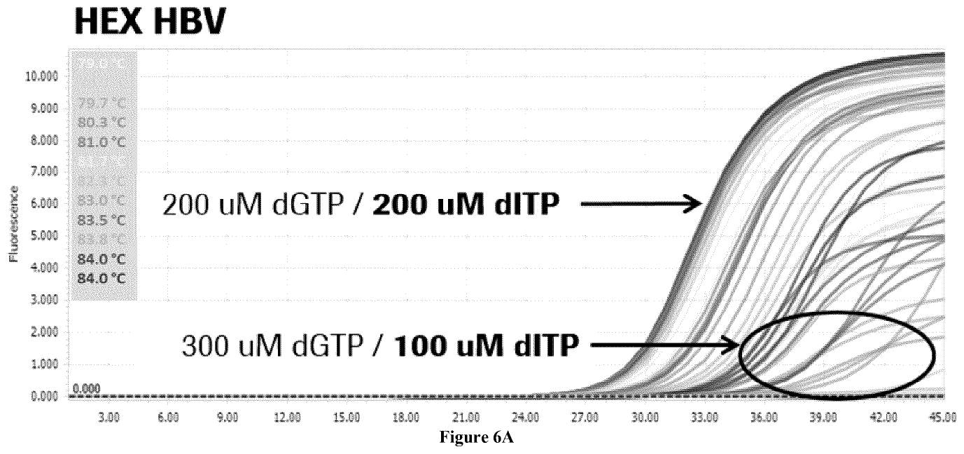

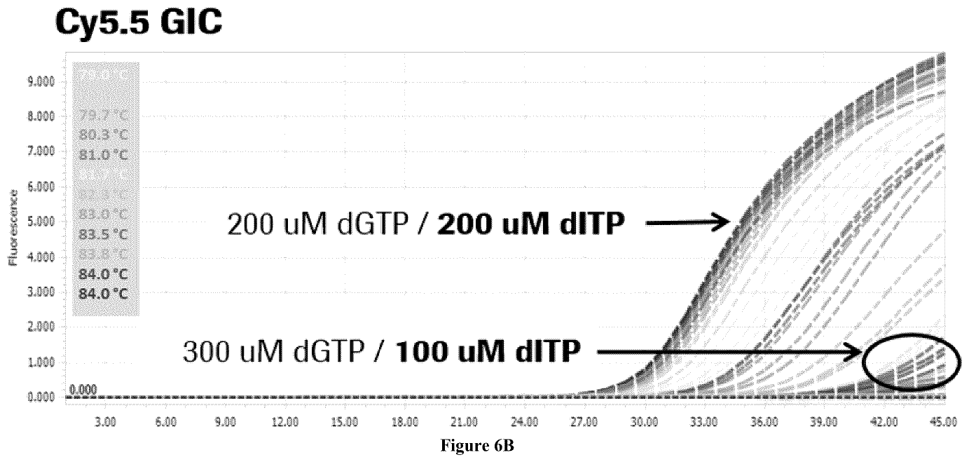

The majority of the evaluations were performed with SYTO 9 dye to examine the effect of dITP on Tm. In this experiment, the HBV and GIC assays were duplexed with TaqMan probes and evaluated on a denaturation temperature gradient of 79 to 84.0° C. Two master mix formulations with 100 M of dITP and 200 μM of dITP were tested on a target pool of HBV Insert 3 (FIG. 6A) and GIC transcripts (FIG. 6B). Both HEX and Cy5.5 TaqMan probes were present in this evaluation.

Results show that there is a considerable performance difference between the two formulations across the denaturation temperature gradient, as shown in FIGS. 6A and 6B. The 200 M dITP concentration recovered the performance at lower denaturation temperatures and was successfully able to reliably detect the HBV RNA target at 79.0° C. which is up to 16° C. lower than the denaturation temperatures utilized in the generic Cobas® 6800/8800 thermal profile (FIG. 6A). A similar trend was observed with the GIC transcript in channel Cy5.5 (FIG. 6B).

### Example 5: Performance of dITP on Clinical HBV Plasma Samples

Following further titration and temperature gradient experiments, the optimal nucleotide concentrations and thermal cycling parameters were determined to be 300 μM dITP (and 100 M dGTP), 78° C. denaturation (during PCR cycling) and UNG deactivation hold, and 55° C. annealing temperature hold (optimization data not shown). The generic Cobas® 6800/8800 thermal profile remained the same other than the specified temperature changes. The finalized dITP thermal cycling profile is listed in Table 5, below.

TABLE 5
Thermal Profiles (+RT Hold) Tested in Experiment.

| | | | | | |
|---|---|---|---|---|---|
| | Cy- | Temperature | Hold time | Acqui- | |
| Step | cles | (° C.) | (hh:mm:ss) | sition | Ramp |

Reactions Plus dITP
(Finalized dITP Thermal Cycling Profile)

| | | | | | |
|---|---|---|---|---|---|
| UNG | 1 | 50 | 0:02:00 | — | 4.4 |
| UNG Deactivation | | 78 | 0:00:05 | — | 4.4 |
| | | 55 | 0:02:00 | — | 2.2 |
| | | 60 | 0:06:00 | — | 4.4 |
| | | 65 | 0:04:00 | — | 4.4 |
| PCR | 5 | 78 | 0:00:05 | — | 4.4 |
| | | 55 | 0:00:30 | — | 2.2 |
| | 45 | 78 | 0:00:05 | — | 4.4 |
| | | 55 | 0:00:25 | Single | 2.2 |
| Cool-down | 1 | 40 | 0:02:00 | — | 2.2 |

Reactions Minus dITP
(Generic cobas ® 6800/8800 Thermal Cycling Profile)

| | | | | | |
|---|---|---|---|---|---|
| UNG | 1 | 50 | 0:02:00 | — | 4.4 |
| UNG Deactivation | | 94 | 0:00:05 | — | 4.4 |
| RT | | 55 | 0:02:00 | — | 2.2 |
| | | 60 | 0:06:00 | — | 4.4 |
| | | 65 | 0:04:00 | — | 4.4 |
| PCR | 5 | 95 | 0:00:05 | — | 4.4 |
| | | 55 | 0:00:30 | — | 2.2 |
| | 45 | 91 | 0:00:05 | — | 4.4 |
| | | 58 | 0:00:25 | Single | 2.2 |
| Cool-down | 1 | 40 | 0:02:00 | — | 2.2 |

The reverse transcription (RT) PCR step in the thermal cycling profile comprises of the three temperature holds of 55, 60, and 65° C. following the UNG Deactivation step listed in Table 4. RT-PCR occurs during the RT step where the synthesis of complementary DNA is created from RNA. The purpose to evaluate with and without RT hold is to compare the DNA (minus RT hold) and the RNA (plus RT hold) amplification. The thermal profiles in Table 4 were tested with and without the RT hold to compare the RNA and DNA amplification in the clinical HBV plasma eluates, described here. Six clinical HBV plasma samples were extracted by the Cobas® 6800/8800 and the Roche High Pure Viral Nucleic Acid Kit. The clinical samples were taken across patients who have or have not undergone treatment. The HBV viral load in these clinical samples ranged from a very high titer of 5.7E9, down to 4.8E4, as shown on Table 6, below.

TABLE 6
List of HBV clinical plasma samples tested.

| Specimen ID | Patient Visit * | Titer |
|---|---|---|
| BCP215625 | Screening | 5.71E+09 |
| BCP217667 | Week 4 | 1.51E+08 |
| BCP218013 | Week 12 | 1.82E+06 |
| BCP243137 | Week 84 | 1.69E+06 |
| BCP218004 | Week 12 | 4.53E+05 |
| BCP243199 | Week 36 | 4.75E+04 |

* Patients undergoing HBV treatment or HBV screening prior to treatment

All samples were evaluated±dITP (optimized concentration of 300 μM) and RT hold to assess the RNA and DNA amplification in the total nucleic acid purified samples. The dITP-optimized thermal profile (including the 78° C. denaturation/UNG deactivation and 55° C. annealing temperatures) was tested with reactions plus dITP. Reactions minus dITP were tested with the generic Cobas® 6800/8800 generic thermal profile (including the 95° C. and 91° C. denaturation holds) for comparison.

TABLE 7
Clinical HBV Plasma Samples Average Cps

| | Minus dITP | | | Plus dITP | | |
|---|---|---|---|---|---|---|
| Sample # | +RT | −RT | ΔCp | +RT | −RT | ΔCp |

cobas ® 6800/8800 Extracted HBV Plasma

| | | | | | | |
|---|---|---|---|---|---|---|
| BCP215625 | 15.1 | 16.2 | 1.1 | 19.0 | 23.2 | 4.2 |
| BCP217667 | 19.6 | 21.8 | 2.1 | 22.1 | 28.8 | 6.7 |
| BCP218013 | 22.1 | 24.1 | 2.0 | 25.4 | 31.8 | 6.3 |
| BCP243137 | 24.0 | 25.8 | 1.8 | 28.1 | 33.6 | 5.5 |
| BCP218004 | 23.0 | 25.0 | 2.0 | 26.7 | 33.2 | 6.5 |
| BCP243199 | 26.8 | 29.2 | 2.4 | 32.3 | 40.0 | 7.7 |

Roche High Pure Viral Nucleic Acid Kit Extracted HBV Plasma

| | | | | | | |
|---|---|---|---|---|---|---|
| BCP215625 | 18.6 | 19.7 | 1.1 | 27.7 | 36.1 | 8.5 |
| BCP217667 | 24.8 | 26.2 | 1.4 | 32.2 | 40.0 | 7.8 |
| BCP218013 | 27.1 | 28.9 | 1.8 | 34.8 | 40.0 | 5.2 |
| BCP243137 | 28.7 | 30.9 | 2.1 | 34.7 | 40.0 | 5.3 |
| BCP218004 | 28.5 | 30.5 | 1.9 | 36.9 | 40.0 | 3.1 |
| BCP243199 | 30.4 | 33.3 | 2.8 | 35.6 | 40.0 | 4.4 |

Minus dITP Average ΔCp: 1.9
Plus dITP Average ΔCp: 6.2
Minus dITP Average ΔCp: 1.9
Plus dITP Average ΔCp: 5.7

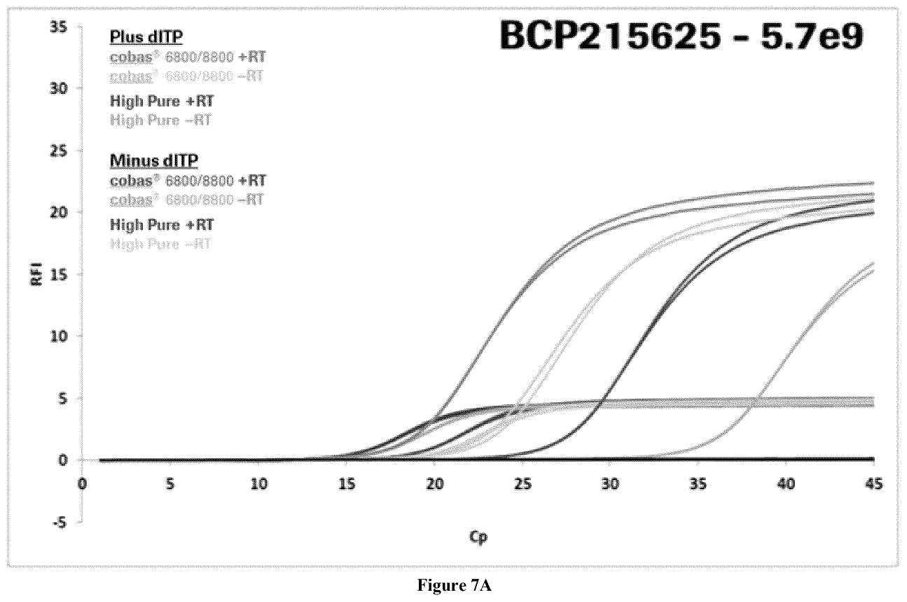

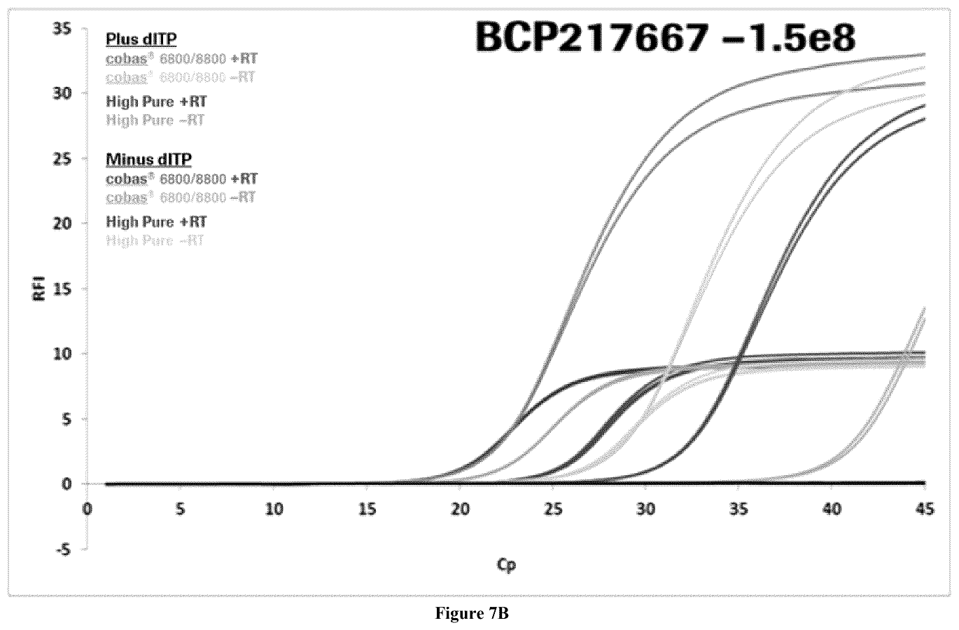

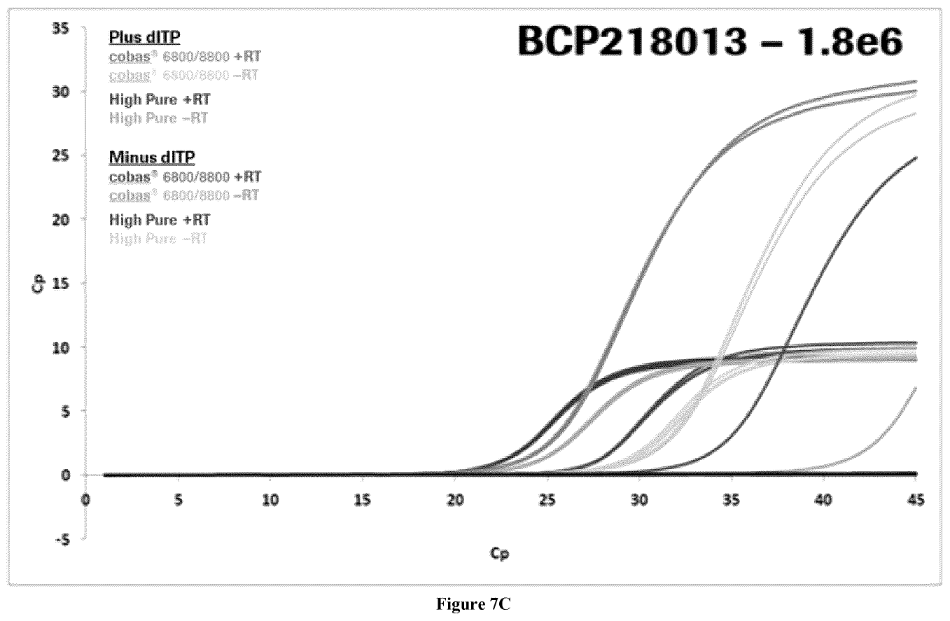

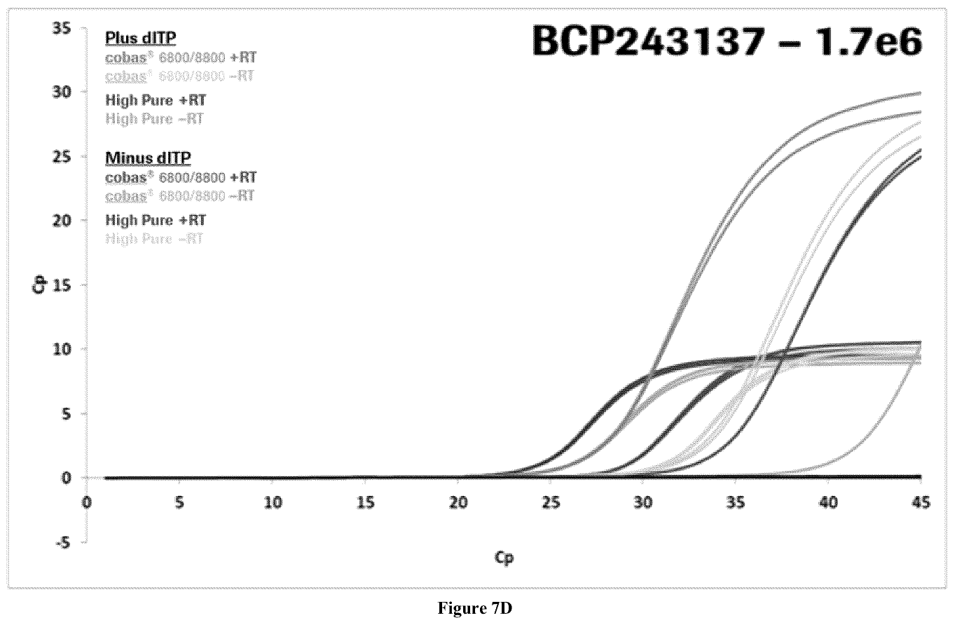

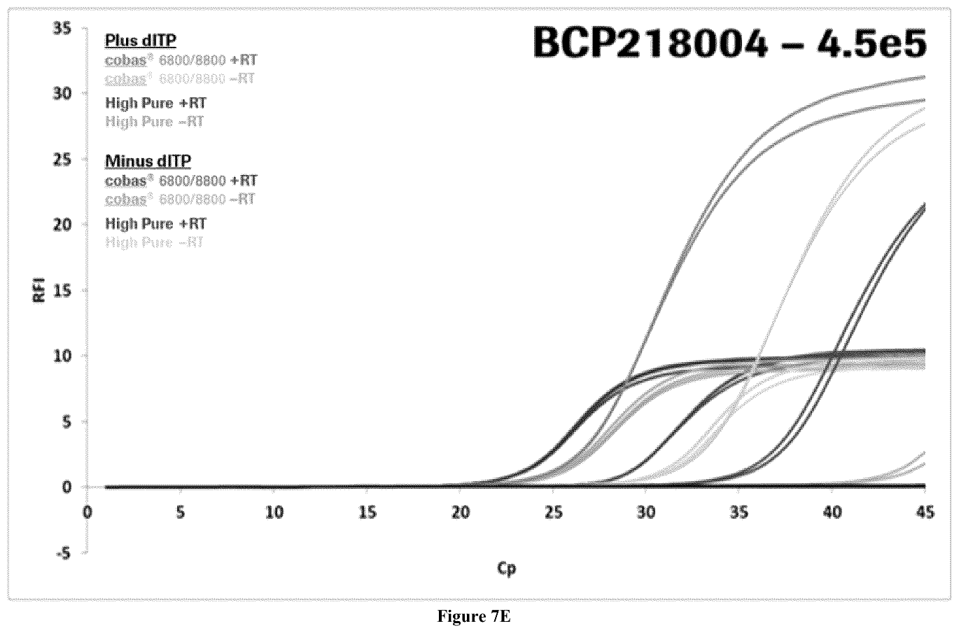

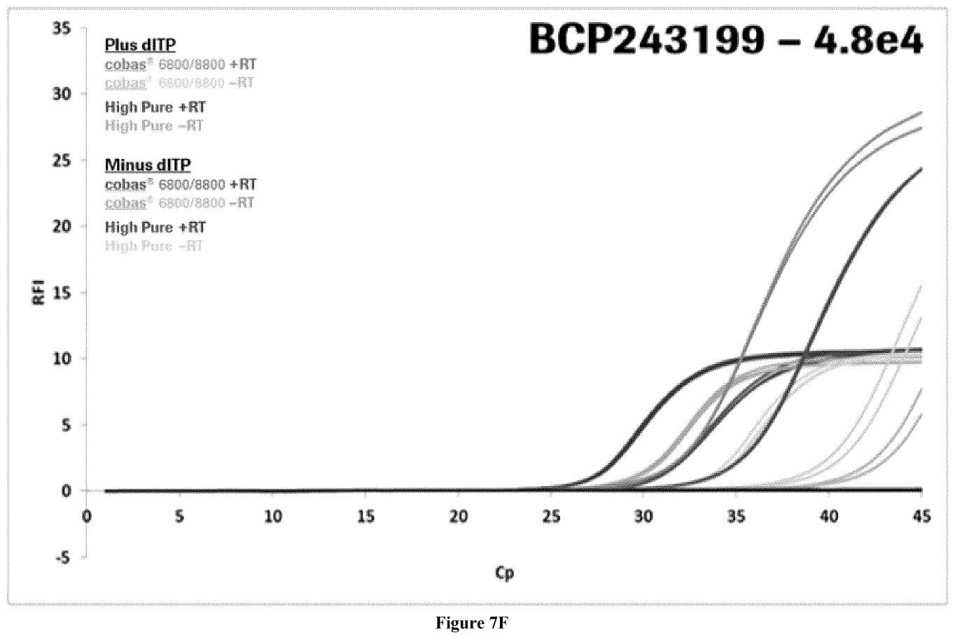

Results show that across all the clinical plasma samples tested (six samples total), the reactions plus dITP generated significantly higher fluorescence signals and considerably larger ΔCp between the reactions±RT hold, as shown in FIGS. 7A-7F. Table 6 shows clinical HBV plasma sample average Cps. Table 6 reveals that plus dITP reactions generate average ΔCps of 6.2 and 5.7, which are notably larger in comparison to the reactions minus dITP (with an average ΔCp of 1.9), across both extraction methods, as shown in Table 7.

This demonstrates an improvement in HBV RNA specificity and indicates preferential and/or selective amplification of RNA target versus DNA in the presence of dITP. The reactions minus dITP generate minimal Cp delay between ±RT hold which suggests that the HBV DNA amplification efficiency is equivalent or close to that of the HBV RNA target.

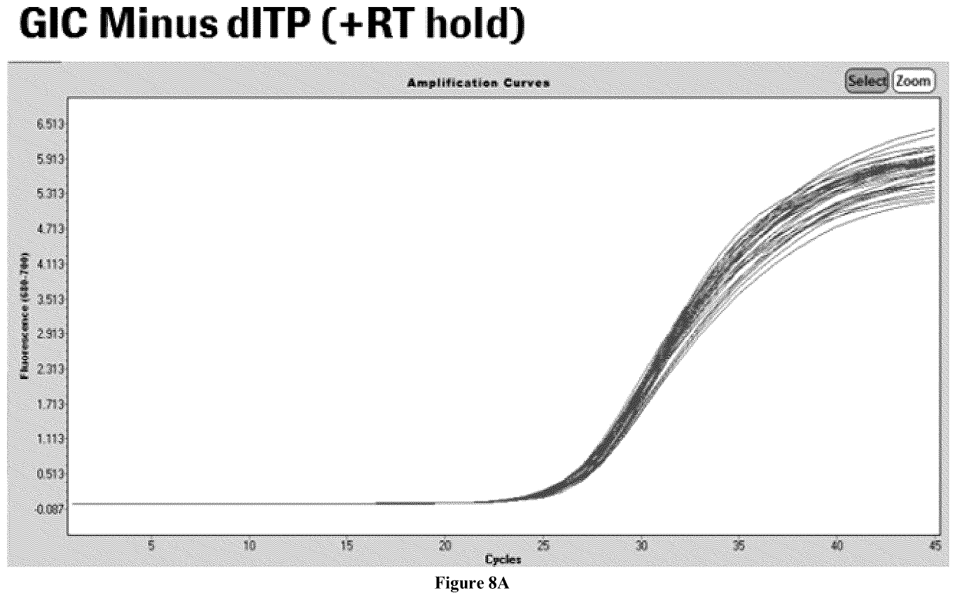

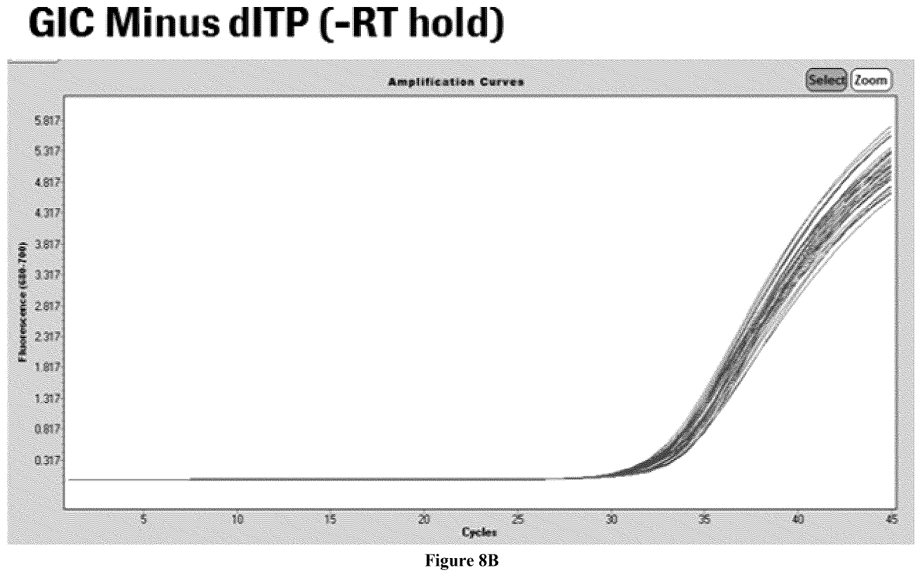

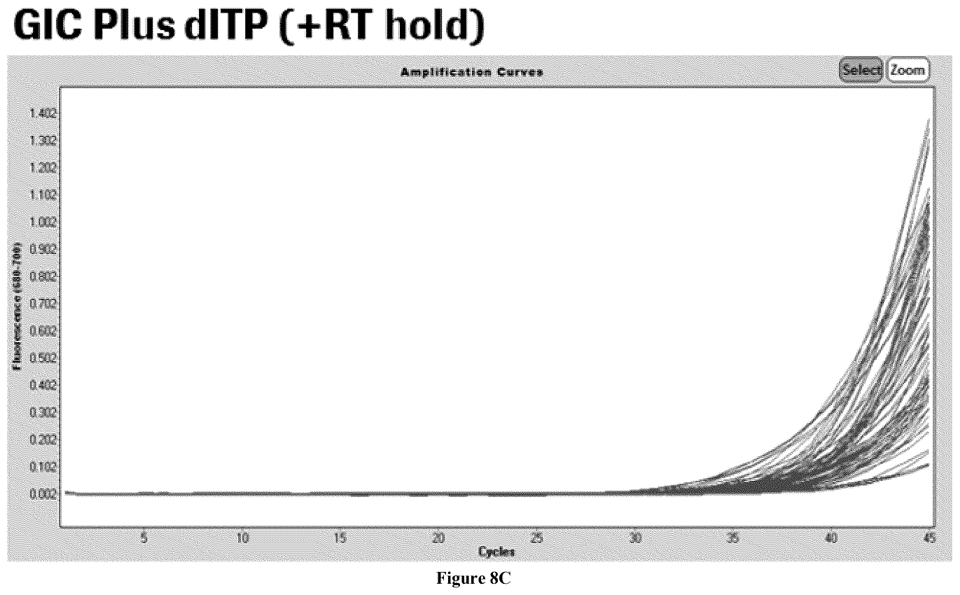

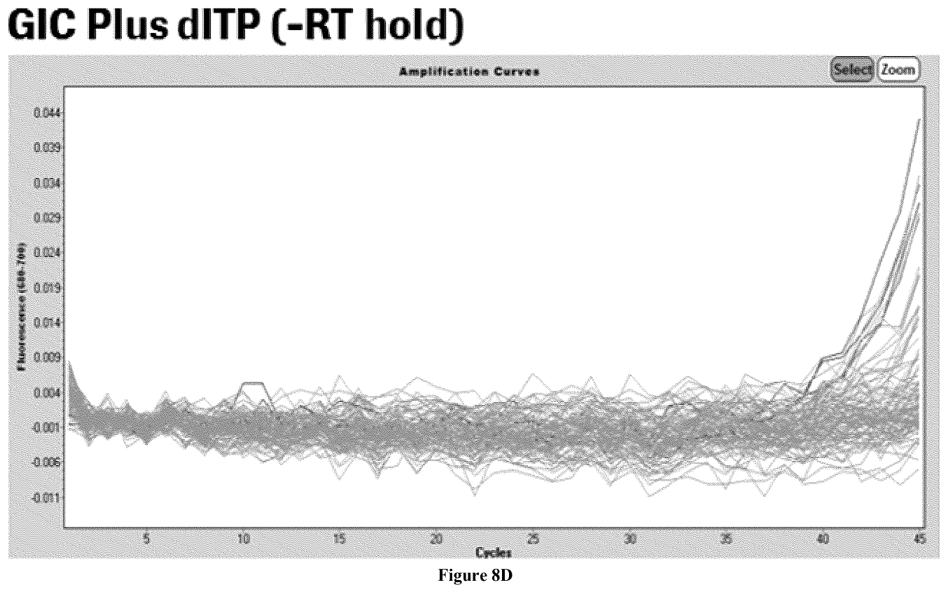

In the Cy5.5 channel, the reactions plus dITP generally delayed the GIC amplification curves and generated low fluorescence signals. An adjustment of the GIC target concentration may be necessary with the utilization of dITP. As expected, the GIC transcript plus dITP/minus RT hold do not generate any Cp calls, as shown on the in FIG. 8D. The reactions minus dITP/minus RT hold generate amplification curves and Cp calls. This may be due to the presence of RT activity in these reactions even in the absence of the RT hold. Thermal cycling at higher temperatures could potentially be contributing to this outcome.

Taken together, these Examples showed that preferential and/or selective amplification of HBV RNA target instead of HBV DNA was demonstrated with the HBV Region 7 assay by integrating dITP-chemistry and changing the thermal cycling profile with limited alterations of the Cobas® 6800/8800 master mix formulation. These Examples demonstrate the potential to apply this method for selective RNA amplification of RNA targets that cannot be easily discerned from native DNA targets or targeted by exploiting the poly-A tail or intron/exon junctions.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method for selectively detecting and/or quantitating one or more target RNA over DNA in a sample, wherein the method comprises the following steps:
(a) providing a sample;
(b) providing one or more polymerases;
(c) providing dNTPs, wherein the dNTPs comprise at least dITP;
(d) performing an amplification step, wherein the amplification step comprises contacting the sample with one or more set of primers specific for the target RNA;
(e) performing a hybridizing step, wherein the hybridizing step comprises contacting the amplification product, if the target RNA is present in the sample, with one or more set of probes; and
(f) performing a detecting step, wherein the detecting step comprises detecting the presence or absence of the target RNA, wherein the presence of the amplification product is indicative of the presence of the target RNA in the sample, and wherein the absence of the amplification product is indicative of the absence of the target RNA in the sample; and
wherein during the amplification step, the one or more polymerases amplify the target RNA, if present in the sample, by incorporating the dITP into the amplification product, thereby selectively detecting and/or quantitating target RNA as compared to DNA in a sample.

2. The method of claim 1, wherein the dNTPs further comprise dATP, dTTP, dCTP, and dGTP.

3. The method of claim 2, wherein the ratio of dITP:dGTP is 3:1, 1:1, or 1:3.

4. The method of claim 3, wherein the ratio of dITP:dGTP is 3:1.

5. The method of claim 2, wherein the dNTPs comprises equal amounts of:
(i) dATP;
(ii) dTTP;
(iii) dCTP; and
(iv) dGTP+dITP.

6. The method of claim 5, wherein the ratio of dITP:dGTP is 3:1, 1:1, or 1:3.

7. The method of claim 6, wherein the ratio of dITP:dGTP is 3:1.

8. The method of claim 1, wherein the sample contains both RNA and DNA.

9. The method of claim 1, wherein the sample is a biological sample.

10. The method of claim 9, wherein the biological sample is blood, plasma, or urine.

11. The method of claim 1, wherein the one or more polymerase is a DNA polymerase.

12. The method of claim 1, wherein the presence of the dITP results in a reduction in the melting temperature (Tₘ) of the amplification product.

13. The method of claim 1, wherein the target RNA is Hepatitis B Virus (HBV) RNA.

14. The method of any one of claims 1-13, wherein the one or more set of primers and the one or more set of probes hybridize to a nucleic acid sequence comprising SEQ ID NO:4.

15. The method of claim 14, wherein the one or more set of primers comprise primers having a nucleic acid sequence of SEQ ID NOs:1 and 2, and wherein the one or more set of probes comprise a probe having a nucleic acid sequence of SEQ ID NO:3.

16. The method of claim 13, wherein the HBV RNA is HBV pre-genomic RNA (pgRNA).

17. The method of claim 16, wherein the HBV pgRNA is a surrogate for HBV covalently closed circular DNA (cccDNA).

18. The method of claim 17, wherein the amount of HBV pgRNA quantitated is a factor considered for a treatment decision regarding a patient from whom the sample originates.

19. A kit for selectively detecting and/or quantitating one or more target RNA over DNA in a sample, wherein the kit comprises:
(a) one or more polymerases;
(b) dNTPs, wherein the dNTPs comprise at least dITP;
(c) one or more set of primers specific for the target RNA; and
(d) one or more set of probes.

20. The kit of claim 19, wherein the dNTPs further comprise dATP, dTTP, dCTP, and dGTP.

21. The kit of claim 20, wherein the ratio of dITP:dGTP is 3:1, 1:1, or 1:3.

22. The kit of claim 21, wherein the ratio of dITP:dGTP is 3:1.

23. The kit of claim 20, wherein the dNTPs comprises equal amounts of:
(i) dATP;
(ii) dTTP;
(iii) dCTP; and
(iv) dGTP+dITP.

24. The kit of claim 23, wherein the ratio of dITP:dGTP is 3:1, 1:1, or 1:3.

25. The kit of claim 24, wherein the ratio of dITP:dGTP is 3:1.

26. The kit of claim 19, wherein the sample contains both RNA and DNA.

27. The kit of claim 19, wherein the sample is a biological sample.

28. The kit of claim 27, wherein the biological sample is blood, plasma, or urine.

29. The kit of claim 19, wherein the one or more polymerase is a DNA polymerase.

30. The kit of claim 19, wherein the presence of the dITP results in a reduction in the melting temperature (Tₘ) of the amplification product.

31. The kit of claim 19, wherein the target RNA is Hepatitis B Virus (HBV) RNA.

32. The kit of any one of claims 19-31, wherein the one or more set of primers and the one or more set of probes hybridize to a nucleic acid sequence comprising SEQ ID NO:4.

33. The kit of claim 32, wherein the one or more set of primers comprise primers having a nucleic acid sequence of SEQ ID NOs:1 and 2, and wherein the one or more set of probes comprise a probe having a nucleic acid sequence of SEQ ID NO:3.

34. The kit of claim 31, wherein the HBV RNA is HBV pre-genomic RNA (pgRNA).

35. The kit of claim 34, wherein the HBV pgRNA is a surrogate for HBV covalently closed circular DNA (cccDNA).

36. The kit of claim 35, wherein the amount of HBV pgRNA quantitated is a factor considered for a treatment decision regarding a patient from whom the sample originates.

37. A method for selectively detecting and/or quantitating one or more target HBV RNA over DNA in a sample, wherein the method comprises the following steps:
(a) providing a sample;
(b) providing one or more polymerases;
(c) providing dNTPs, wherein the dNTPs comprise at least dITP;
(d) performing an amplification step, wherein the amplification step comprises contacting the sample with one or more set of primers specific for the target HBV RNA, wherein the one or more set of primers comprise primers having a nucleic acid sequence of SEQ ID NOs:1 and 2;
(e) performing a hybridizing step, wherein the hybridizing step comprises contacting the amplification product, if the target HBV RNA is present in the sample, with one or more set of probes, wherein the one or more set of probes comprise a probe having a nucleic acid sequence of SEQ ID NO:3; and
(f) performing a detecting step, wherein the detecting step comprises detecting the presence or absence of the target HBV RNA, wherein the presence of the amplification product is indicative of the presence of the target HBV RNA in the sample, and wherein the absence of the amplification product is indicative of the absence of the target HBV RNA in the sample; and
wherein during the amplification step, the one or more polymerases amplify the target HBV RNA, if present in the sample, by incorporating the dITP into the amplification product, thereby selectively detecting and/or quantitating target RNA as compared to DNA in a sample.

38. The method of claim 37, wherein the dNTPs further comprise dATP, dTTP, dCTP, and dGTP.

39. The method of claim 38, wherein the ratio of dITP:dGTP is 3:1, 1:1, or 1:3.

40. The method of claim 39, wherein the ratio of dITP:dGTP is 3:1.

41. The method of claim 38, wherein the dNTPs comprises equal amounts of:
(i) dATP;
(ii) dTTP;
(iii) dCTP; and
(iv) dGTP+dITP.

42. The method of claim 41, wherein the ratio of dITP:dGTP is 3:1, 1:1, or 1:3.

43. The method of claim 42, wherein the ratio of dITP:dGTP is 3:1.

44. The method of claim 37, wherein the sample contains both RNA and DNA.

45. The method of claim 37, wherein the sample is a biological sample.

46. The method of claim 45, wherein the biological sample is blood, plasma, or urine.

47. The method of claim 37, wherein the one or more polymerase is a DNA polymerase.

48. The method of claim 37, wherein the presence of the dITP results in a reduction in the melting temperature (Tₘ) of the amplification product.

49. The method of claim 37, wherein the one or more set of primers and the one or more set of probes hybridize to a nucleic acid sequence comprising SEQ ID NO:4.

50. The method of claim 37, wherein the HBV RNA is HBV pre-genomic RNA (pgRNA).

51. The method of claim 50, wherein the HBV pgRNA is a surrogate for HBV covalently closed circular DNA (cccDNA).

52. The method of claim 51, wherein the amount of HBV pgRNA quantitated is a factor considered for a treatment decision regarding a patient from whom the sample originates.


SEQUENCE LISTING (8 sequences)

SEQ ID NO: 1 - DNA, 25 bp, Artificial Sequence
  Forward Primer
  modified_base (4)..(4): C-5 propynyl-dU
  modified_base (13)..(13): C-5 propynyl-dU
  modified_base (16)..(16): C-5 propynyl-dU
  modified_base (19)..(19): C-5 propynyl-dU
  modified_base (22)..(22): C-5 propynyl-dU
  modified_base (25)..(25): t-Butyl Benzyl-dA
acttccggaaactactgttgttaga

SEQ ID NO: 2 - DNA, 24 bp, Artificial Sequence
  Reverse Primer
  modified_base (8)..(8): C-5 propynyl-dU
  modified_base (14)..(14): C-5 propynyl-dU
  modified_base (18)..(18): 5-Methyl-dC
  modified_base (21)..(21): 5-Methyl-dC
  modified_base (24)..(24): t-Butyl Benzyl-dC
cccgagattgagatcttctgcgac

SEQ ID NO: 3 - DNA, 32 bp, Artificial Sequence
  Probe
  misc_feature (0)..(1): HEX-Thr
  modified_base (2)..(2): 5-Methyl-dC
  modified_base (3)..(3): C-5 propynyl-dU
  misc_feature (8)..(9): BHQ-2
  modified_base (14)..(14): 5-Methyl-dC
  misc_feature (32)..(33): Phosphate
cctagaagaagaactccctcgcctcgcagacg

SEQ ID NO: 4 - DNA, 113 bp, Artificial Sequence
  HBV Amplicon
acttccggaaactactgttgttagacgacgaggcaggtcccctagaagaagaactccctcgcctcgcagacgaaggtctcaatcgccgcgtcgcagaagatctcaatctcggg

SEQ ID NO: 5 - DNA, 25 bp, Artificial Sequence
  Forward Primer
  modified_base (25)..(25): t-Butyl Benzyl-dA
acaaccgcgccatacatgtcaagaa

SEQ ID NO: 6 - DNA, 24 bp, Artificial Sequence
  Reverse Primer
  modified_base (24)..(24): t-Butyl Benzyl-dA
gtcgggccgcttatacagtaccaa

SEQ ID NO: 7 - DNA, 33 bp, Artificial Sequence
  Probe
  misc_feature (0)..(1): Cy5.5
  misc_feature (11)..(12): BHQ-2
  misc_feature (33)..(34): Phosphate
tgcgcgtcccgttttgatacttcgtaacggtgc

SEQ ID NO: 8 - DNA, 60 bp, Artificial Sequence
  Amplicon
acaaccgcgccatacatgtcaagaatgaagtgggcgaacgctagaaaactgacgccagca